(19) United States
(12) Patent Application Publication
(10) Pub. No.: US 2015/0031701 A1
(43) Pub. Date: Jan. 29, 2015

(54) COMPOSITIONS AND METHODS FOR PREVENTING AND TREATING MUCOSITIS AND WEIGHT LOSS

(71) Applicant: ST IP Holding AG, Zug (CH)

(72) Inventor: Patrick T. Prendergast, Byrock (AU)

(21) Appl. No.: 14/452,965

(22) Filed: Aug. 6, 2014

Related U.S. Application Data
(63) Continuation of application No. 13/899,967, filed on May 22, 2013, now abandoned.
Application No. 13/899,967 is a continuation of application No. 12/450,087, filed on Oct. 26, 2009, now abandoned.
(60) Provisional application No. 60/906,442, filed on Mar. 12, 2007.

(30) Foreign Application Priority Data
Mar. 12, 2007 (GB) 0704718.6

(51) Int. Cl. A61K 31/497; A61K 45/06; A61K 31/4985

(52) U.S. Cl. CPC A61K 31/497; A61K 31/4985; A61K 45/06
USPC 514/250; 514/255.05

(57) ABSTRACT

Pharmaceutical compositions comprising 5-[2-pyrazinyl]-4-methyl-1,2-3-thione or an analogue, derivative, metabolite, prodrug, solvate or a pharmaceutically acceptable salt thereof; and a pharmaceutical carrier, which promotes association with the outer wall of a subject's digestive tract are described as well as their uses in treating and preventing mucositis and cachexia.

### FIELD OF THE INVENTION

The present invention provides compositions and methods for the prophylaxis and/or treatment of mucositis. More specifically, the present invention provides compositions comprising oltipraz (5-[2-pyrazinyl]-4-methyl-1,2-3-thione) or analogues or derivatives thereof, and/or N⁶ isopentenyl adenosine or analogues or derivatives thereof, and their use in the prevention and treatment of mucositis. Also provided are compositions and methods for reducing weight loss in subjects undergoing radiotherapy and for prophylaxis and treatment of cachexia.

### BACKGROUND TO THE INVENTION

Mucositis is an inflammatory condition of the mucous membranes or mucosa lining the digestive tract. The condition is caused by a breakdown of the mucosa, which results in the formation of ulcerative lesions. These lesions can be extremely painful and can occur at sites in the alimentary tract from the oral cavity to the anus, including the oesophagus, stomach, small intestine, colon and rectum.

Mucositis is a common side effect of chemotherapy or radiotherapy. The mucosa of the mouth and digestive tract are sensitive to both chemotherapy and radiotherapy. The chemotherapeutic agents used to treat cancerous conditions adversely affect normal cells, in particular those which have high turnover rates, such as the cells of the oral epithelial tissues. These radiation therapy treatments cause cell death, which results in the mucosal lining becoming thin, sloughed off and then red, inflamed and ulcerated. Patients undergoing chemotherapy usually become symptomatic within four to five days of commencing treatment. Mucositis associated with radiotherapy generally presents within 14 days of treatment, with the symptoms persisting for 6 to 8 weeks.

The pathophysiology of mucositis can be divided into five stages including an initiation stage, a message generation stage, a signalling and amplification stage, an ulceration stage and a healing stage. The different stages are caused by different cytokines. The initiation stage follows chemotherapy or radiotherapy, which results in the production of free radicals which cause DNA damage. In turn, transcription factors, such as NF-kB, are produced that upregulate inflammatory cytokine production. This inflammation, which is mediated by cytokines such as IL-1 and TNF-alpha, causes the ulceration stage.

The main clinical manifestations of mucositis include esophagitis (inflammation of the esophagus), dysphagia (difficulty in swallowing), odynophagia (painful swallowing), substernal chest pain (in radiation induced mucositis) and retrosternal chest pain (caused by chemotherapy).

There are no effective treatments for mucositis. Current treatments are generally palliative and include maintaining a high level of oral hygiene, the use of topical analgesics, such as lidocaine, and mouthwashes, such as chlorohexidine gluconate. Further therapies include the use of agents which reduce the mucosal absorption of chemotherapy drugs, for example cryotherapy or allopurinol. Other treatments, such as glutamine or beta-carotene, reduce changes in epithelial proliferation. Further treatments include laser therapy and antibiotics, as well as the use of cytokine-based therapies, such as palifermin (brand name Kepivance, Amgen), which is a human keratinocyte growth factor (KGF), and other modulators of inflammation.

None of the currently used therapeutic approaches has proved entirely effective in the prophylaxis or treatment of mucositis. There is therefore a substantial unmet clinical need for therapies which can be used for the effective prophylaxis and treatment of mucositis. Such therapies will be particularly beneficial to patients presenting with cancerous conditions who will undergo, or who are undergoing, cancer therapy such as chemotherapy and/or radiotherapy.

Cachexia is loss of weight, muscle atrophy, fatigue, weakness and significant loss of appetite in someone who is not actively trying to lose weight. It can be a sign of various underlying disorders, such as cancer, certain infectious diseases (e.g. tuberculosis, AIDS) and some autoimmune disorders, or addiction to drugs such as amphetamines or cocaine.

The inventor has surprisingly identified novel compositions and methods for the prevention or treatment of mucositis in subjects who are undergoing radiation therapy. Specifically, the inventor has identified a number of non-steroidal compounds which have been unexpectedly shown to have utility in the prophylaxis and treatment of mucositis. The inventor has also identified compositions having utility in the prophylaxis and treatment of cachexia and in reducing weight loss in subjects undergoing cancer treatment by radiotherapy.

### SUMMARY OF THE INVENTION

According to a first aspect of the present invention there is provided a method for the prophylaxis and/or treatment of mucositis, the method comprising the steps of:
providing a therapeutically effective amount of a composition comprising at least one compound selected from the group consisting of 5-[2-pyrazinyl]-4-methyl-1,2-3-thione (oltipraz) or an analogue, derivative, metabolite, prodrug, solvate or pharmaceutically acceptable salt thereof; and
administering the composition to a subject.

According to a second aspect of the present invention there is provided a composition comprising at least one compound selected from the group consisting of 5-[2-pyrazinyl]-4-methyl-1,2-3-thione (oltipraz) or an analogue, derivative, metabolite, prodrug, solvate or pharmaceutically acceptable salt thereof.

According to a third aspect of the present invention there is provided the use of a composition comprising at least one compound selected from the group consisting of 5-[2-pyrazinyl]-4-methyl-1,2-3-thione (oltipraz) or an analogue, derivative, metabolite, prodrug, solvate or pharmaceutically acceptable salt thereof in the preparation of a medicament for the treatment and/or prophylaxis of mucositis.

According to a fourth aspect of the present invention there is provided a composition for use in the prevention and/or treatment of mucositis, the composition comprising 5-[2-pyrazinyl]-4-methyl-1,2-3-thione (oltipraz) or an analogue, derivative, metabolite, prodrug, solvate or pharmaceutically acceptable salt thereof.

According to a fifth aspect of the present invention there is provided a pharmaceutical composition comprising at least one compound selected from the group consisting of 5-[2-pyrazinyl]-4-methyl-1,2-3-thione (oltipraz) or an analogue, derivative, metabolite, prodrug, solvate or pharmaceutically acceptable salt thereof along with a pharmaceutically acceptable carrier.

In certain embodiments of the above mentioned aspects of the invention, the metabolite of 5-[2-pyrazinyl]-4-methyl-1,2-3-thione is the pyrrolopyrazine derivative metabolite 3 (also known as M3).

In certain embodiments, the analogue of 5-[2-pyrazinyl]-4-methyl-1,2-3-thione is a compound of the 1,2-dithiol-3-thione class, for example, anethole trithione ((5-(p-methoxyphenyl)-3H-1,2-dithiole-3-thio) (also known as anetol tritiona or SONICUR™).

In certain embodiments, the 5-[2-pyrazinyl]-4-methyl-1,2-3-thione compound is 1,2-dithiole-3-thione (D3T) or an analogue thereof. Typically the 1,2-dithiole-3-thione analogue has the following formula:

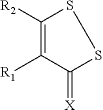

wherein:
in the case of 5-substituted analogues:
   R₁ is H, R₂ is phenyl and X is S,
   R₁ is H, R₂ is 4-methoxyphenyl and X is S,
   R₁ is H, R₂ is 2-pyrazinyl and X is O or
   R₁ is H, R₂ is 2-(5,6-dimethyl)pyrazinyl and X is S;
in the case of 5-substituted-4-methyl analogues:
   R₁ is CH₃, R₂ is 2-pyridyl and X is S,
   R₁ is CH₃, R₂ is 3-pyridyl and X is S,
   R₁ is CH₃, R₂ is 4-pyridyl and X is S,
   R₁ is CH₃, R₂ is 3-pyridazinyl and X is S,
   R₁ is CH₃, R₂ is 2-thiofuranyl and X is S or
   R₁ is CH₃, R₂ is 2-(2-pyrazinyl)ethylene and X is S;
in the case of 4-substituted-5-(2-pyrazinyl) analogues:
   R₁ is CH₃, R₂ is 2-pyrazinyl and X is S,
   R₁ is CH₃, R₂ is 2-pyrazinyl and X is O,
   R₁ is CH₂OH, R₂ is 2-pyrazinyl and X is S,
   R₁ is CH₂CH₃, R₂ is 2-pyrazinyl and X is S or
   R₁ is (CH₂)₃CH₃, R₂ is 2-pyrazinyl and X is S;
in the case of miscellaneous analogues:
   R₁ is CO₂C₂H₅, R₂ is 2-pyridyl and X is S,
   R₁ is CO₂C₂H₅, R₂ is 4-pyridyl and X is S,
   R₁ is C₁, R₂ is [4-(2-propyl)phenyl]amino and X is S,
   R₁ is C₁, R₂ is [4-(2-propyl)phenyl]amino and X is O,
   R₁ is CH₂CO₂C₂H₅, R₂ is 5-pyrimidyl and X is S,
   R₁ is CH₂CON[CH(CH₃)₂]₂, R₂ is 5-pyrimidyl and X is S,
   R₁ is phenethyl, R₂ is 3-pyridazinyl and X is S,
   R₁ is H, R₂ is 4-pyridyl and X is N—O—(CH₂)₃N(CH₃)₂ or
   R₁ is (CH₂)₃CH₃, R₂ is 3-(6-dimethylamino)pyridazinyl and X is S.

In certain further embodiments, R₁ is fluorine or bromine, and R₂ and X are selected from the substituents listed above.

In certain embodiments, the compound of the invention is co-administered along with, or formulated with, carboxymethyl cellulose (CMC).

The inventor has surprisingly identified that administering 5-[2-pyrazinyl]-4-methyl-1,2-3-thione along with carboxymethyl cellulose results in a marked reduction in toxicity associated with 5-[2-pyrazinyl]-4-methyl-1,2-3-thione administration. Specifically, the inventor has identified that when formulated with CMC, 5-[2-pyrazinyl]-4-methyl-1,2-3-thione can be administered to a subject in an amount of up to 2000 mg/kg without significant toxicity resulting. Without wishing to be bound by theory, it is hypothesised that when formulated with carboxymethyl cellulose, 5-[2-pyrazinyl]-4-methyl-1,2-3-thione is not absorbed into the bloodstream but becomes associated with the outer wall of the digestive tract, this resulting in an effective lining of the digestive tract, which serves to protect against damage, such as gastrointestinal damage.

In certain embodiments, the 5-[2-pyrazinyl]-4-methyl-1,2-3-thione compound is administered with, or formulated with, a sulphur-containing amino acid such as cysteine or an analogue, derivative, salt or solvate thereof.

Oral administration of 5-[2-pyrazinyl]-4-methyl-1,2-3-thione with cysteine has been shown to result in a marked increase in both the extent and rate of 5-[2-pyrazinyl]-4-methyl-1,2-3-thione bioavailability (Hassan M. Ali et al., 1984; Chemotherapy 30: 255-261).

In certain embodiments, the 5-[2-pyrazinyl]-4-methyl-1,2-3-thione compound is administered with, or formulated with, a chemotherapeutic agent which may be selected from the group consisting of, but not limited to, cisplatin, dexamethasone and 5-fluorouracil.

The inventor has also surprisingly identified that cytokinin compounds have utility in the prophylaxis and/or treatment and/or amelioration of mucositis or of at least one symptom thereof. Accordingly the present invention further extends to methods, compositions and uses of cytokinin compounds in the treatment, amelioration and/or prophylaxis of mucositis.

According to a further aspect of the present invention there is provided a method for the prophylaxis and/or treatment of mucositis, the method comprising the steps of:
providing a therapeutically effective amount of a composition comprising at least one cytokinin compound or a pharmaceutically acceptable salt or solvate thereof; and
administering the composition to a subject in need of such treatment.

According to a yet further aspect of the invention there is provided a composition comprising at least one cytokinin compound or a pharmaceutically acceptable salt or solvate thereof.

According to a still further aspect of the present invention there is provided the use of a composition comprising at least one cytokinin compound or a pharmaceutically acceptable salt or solvate thereof in the preparation of a medicament for the treatment and/or prophylaxis of mucositis.

According to a yet further aspect of the present invention there is provided a composition for use in the prevention or treatment or mucositis, the composition comprising at least one cytokinin compound or a pharmaceutically acceptable salt or solvate thereof.

According to a yet further aspect of the present invention there is provided a pharmaceutical composition comprising at least one cytokinin compound or a pharmaceutically acceptable salt or solvate thereof along with at least one pharmaceutically acceptable carrier or diluent.

In certain embodiments, the cytokinin compound is N⁶-isopentenyl adenosine (IPA) or an analogue, derivative, metabolite, prodrug, solvate or salt thereof.

In further embodiments, the cytokinin compound is N⁶-benzyl adenosine or an analogue, derivative, metabolite, prodrug, solvate or salt thereof.

In still further embodiments, the cytokinin compound is selected from the group comprising, but not limited to, kinetin, zeatin and benzyl adenine. In particular, the cytokinin compound may include 6-(substituted amino) purines, including kinetin (6-(furfuryl)aminopurine), zeatin (6-(3-hydroxymethyl, 3-methylallyl), aminopurine, 6-(3,3-dimethylallyl)amino-purine, 6-(benzyl)aminopurine, 6-(phenyl)aminopurine, 6-(n-alkyl) aminopurine, wherein the alkyl group has 4, 5 or 6 carbon atoms, and 6-(cyclohexyl) methylaminopurine. In certain embodiments, the 6-(substituted amino)purine cytokinin may be combined, at a concentration of between about 0.01% (w/v) and about 0.5% (w/v), preferably about 0.1% (w/v) with a physiologically acceptable carrier or diluent.

In certain embodiments, the cytokinin compound is administered with, or formulated with, a chemotherapeutic agent, such as cisplatin, dexamethasone or 5-fluorouracil.

In certain embodiments, the cytokinin compound is administered with, or formulated with, carboxymethyl cellulose (CMC).

The inventor has surprisingly identified that administering the cytokinin compound, typically N⁶-isopentenyl adenosine, along with carboxymethyl cellulose results in a marked reduction in toxicity. Specifically, the inventor has identified that when formulated with CMC, the cytokinin compound is safe for administration to a subject in an amount up to 2000 mg/kg, whereas when the cytokinin compound is administered in the absence of CMC, it is expected to cause toxicity in the liver at levels of 50 to 100 mg/kg. Without wishing to be bound by theory, it is hypothesised that when formulated with carboxymethyl cellulose, the cytokinin compound is not absorbed into the bloodstream, but rather lines the digestive tract, thus serving to protect against damage, such as gastrointestinal damage.

According to a further aspect of the present invention, there is provided a combined medicament comprising at least one cytokinin compound or a pharmaceutically acceptable salt or solvate thereof and 5-[2-pyrazinyl]-4-methyl-1,2-3-thione or an analogue, derivative, metabolite, prodrug, solvate or pharmaceutically acceptable salt thereof.

Further provided is a pharmaceutical composition for use in the treatment or prevention of mucositis, said composition comprising said combined medicament along with at least one pharmaceutically acceptable carrier or diluent.

Also provided is the use of the combined medicament or a pharmaceutical composition comprising the same in the performance of the methods of the present invention for the prophylaxis and/or treatment of mucositis.

A further aspect of the invention provides a method for the prophylaxis and/or treatment of mucositis, the method comprising the steps of:
providing a therapeutically effective amount of a composition comprising at least one compound selected from the group consisting of 3H-1,2-dithiole-3-thione, anethole trithione (5-(4-methoxyphenyl)-3H-1,2-dithiole-3-thione), ADT, ADO, 1,2-dithiole-3-thione, 1,2-dithiolane, 1,3-dithiole-2-thione, malotilate, 4-(3,5-diisopropyl-4-hydroxyphenyl)-1,2-dit-hiole-3-thione; 4-(3,5-di-t-butyl-4-hydroxyphenyl)-1,2-dithiole-3-thione; 4-[3,5-bis(l,l-dimethylpropyl)-4-hydroxyphenyl]-1,2-dithi-ole-3-thione; 4-[3,5bis(l,l-dimethylbutyl)-4-hydroxyphenyl]-1,2-dithiole-3-thione; 4-[3,5-bis(1,1,3,3-tetramethylbutyl)-4-hydroxyphenyl]-1,2-dithole-3-thion-e; 4-[3,5-bis(l-methylcyclohexyl)-4-hydroxyphenyl]-1,2-dithiole-3-thione; 4-[3,5-bis(l,l-dimethylbenzyl)-4-hydroxyphenyl]-1,2-dithiole-3-thione; 4-(3t-butyl-4-hydroxy-5-isopropylphenyl)-1,2-dithiole-3-thione; 4-(3t-butyl-4-hydroxy-5-methylphenyl)-1,2-dithiole-3-thione; 4-[3(1,1-dimethylpropyl)-4-hydroxy.-5-isopropylphenyl]-1,2-dithiole-3-thi-one; 4-[3(1,1-dimethylbenzyl)-4-hydroxy-5-isopropylphenyl]-1,2-dithiole-3-thione; 5-benzylthio-4-(3,5-di-t-butyl-4-hydroxyphenyl)-1,2-dithole-3-thione; 5-benzylthio-4-[3,5-bis(l,l-dimethylpropyl)-4-hydroxy-phenyl]-1,2-dithiole-3-thione; 5-hexylthio-4-(3,5-di-t-butyl-4-hydroxyphenyl)-1,2-dithole-3-thione; 5-hexylthio-4-[3,5-bis(l,l-dimethylbutyl)-4-hydroxy-phenyl]-1,2-di-thiole-3-thione; 5-octadecylthio-4-(3,5-di-t-butyl-4-hydroxyphenyl)-1,2-di-thiole-3-thione; 5-octadecylthio-4-[3,5-bis(l,l-dimethylbenzyl)-4-hydroxyp-henyl]-1,2-dithiole-3-thione; 5-allylthio-4-(3,5-di-t-butyl-4-hydroxypheny-l)-1,2-dithiole-3-thione; 5-cyclohexylthio-4-(3,5-di-t-butyl-4-hydroxyphenyl)-1,2-dithiole-3-thione; and 4-(3,5-di-sec-butyl-4-hydroxyphenyl)-1,2-di-thiole-3-thione; and
administering the composition to a subject in need of such treatment.

A yet further aspect of the present invention provides a pharmaceutical composition which comprises at least one compound selected from the group consisting of 3H-1,2-dithiole-3-thione, anethole trithione (5-(4-methoxyphenyl)-3H-1,2-dithiole-3-thione), ADT, ADO, 1,2-dithiole-3-thione, 1,2-dithiolane, 1,3-dithiole-2-thione, malotilate, 4-(3,5-diisopropyl-4-hydroxyphenyl)-1,2-dit-hiole-3-thione; 4-(3,5-di-t-butyl-4-hydroxyphenyl)-1,2-dithiole-3-thione; 4-[3,5-bis(l,l-dimethylpropyl)-4-hydroxyphenyl]-1,2-dithi-ole-3-thione; 4-[3,5bis(l,l-dimethylbutyl)-4-hydroxyphenyl]-1,2-dithiole-3-thione; 4-[3,5-bis(1,1,3,3-tetramethylbutyl)-4-hydroxyphenyl]-1,2-dithole-3-thion-e; 4-[3,5-bis(l-methylcyclohexyl)-4-hydroxyphenyl]-1,2-dithiole-3-thione; 4-[3,5-bis(l,l-dimethylbenzyl)-4-hydroxyphenyl]-1,2-dithiole-3-thione; 4-(3t-butyl-4-hydroxy-5-isopropylphenyl)-1,2-dithiole-3-thione; 4-(3t-butyl-4-hydroxy-5-methylphenyl)-1,2-dithiole-3-thione; 4-[3(1,1-dimethylpropyl)-4-hydroxy.-5-isopropylphenyl]-1,2-dithiole-3-thi-one; 4-[3(1,1-dimethylbenzyl)-4-hydroxy-5-isopropylphenyl]-1,2-dithole-3-thione; 5-benzylthio-4-(3,5-di-t-butyl-4-hydroxyphenyl)-1,2-dithole-3-thion-e; 5-benzylthio-4-[3,5-bis(l,l-dimethylpropyl)-4-hydroxy-phenyl]-1,2-dithole-3-thione; 5-hexylthio-4-(3,5-di-t-butyl-4-hydroxyphenyl)-1,2-dithole-3-thione; 5-hexylthio-4-[3,5-bis(l,l-dimethylbutyl)-4-hydroxy-phenyl]-1,2-d-ithole-3-thione; 5-octadecylthio-4-(3,5-di-t-butyl-4-hydroxyphenyl)-1,2-di-thiole-3-thione; 5-octadecylthio-4-[3,5-bis(l,l-dimethylbenzyl)-4-hydroxyp-henyl]-1,2-dithiole-3-thione; 5-allylthio-4-(3,5-di-t-butyl-4-hydroxypheny-l)-1,2-dithiole-3-thione; 5-cyclohexylthio-4-(3,5-di-t-butyl-4-hydroxyphenyl)-1,2-dithiole-3-thione; and 4-(3,5-di-sec-butyl-4-hydroxyphenyl)-1,2-di-thiole-3-thione along with at least one pharmaceutically acceptable carrier or diluent.

According to a further aspect of the present invention, there is provided a method of reducing and/or preventing weight loss in a subject undergoing cancer treatment, the method comprising the steps of:
providing a therapeutically effective amount of a composition comprising at least one compound selected from the group consisting of 5-[2-pyrazinyl]-4-methyl-1,2-3-thione or an analogue, derivative, metabolite, prodrug, solvate or pharmaceutically acceptable salt thereof; and
administering the composition to the subject.

According to a further aspect of the present invention there is provided the use of a composition comprising at least one compound selected from the group consisting of 5-[2-pyrazinyl]-4-methyl-1,2-3-thione or an analogue, derivative, metabolite, prodrug, solvate or pharmaceutically acceptable salt thereof in the preparation of a medicament for reducing and/or preventing weight loss in a subject undergoing cancer treatment.

According to a further aspect of the present invention there is provided a composition for use in reducing and/or preventing weight loss in a subject undergoing cancer treatment, the composition comprising 5-[2-pyrazinyl]-4-methyl-1,2-3-thione or an analogue, derivative, metabolite, prodrug, solvate or pharmaceutically acceptable salt thereof.

In certain embodiments, the cancer treatment is chemotherapy, radiotherapy or a combination thereof.

According to a further aspect of the present invention, there is provided a method of prophylaxis and/or treatment of cachexia, the method comprising the steps of:
providing a therapeutically effective amount of a composition comprising at least one compound selected from the group consisting of 5-[2-pyrazinyl]-4-methyl-1,2-3-thione or an analogue, derivative, metabolite, prodrug, solvate or pharmaceutically acceptable salt thereof; and
administering the composition to a subject in need of such treatment.

According to a further aspect of the present invention there is provided the use of a composition comprising at least one compound selected from the group consisting of 5-[2-pyrazinyl]-4-methyl-1,2-3-thione or an analogue, derivative, metabolite, prodrug, solvate or pharmaceutically acceptable salt thereof in the preparation of a medicament for the prophylaxis and/or treatment of cachexia.

According to a further aspect of the present invention there is provided a composition for use in the prophylaxis and/or treatment of cachexia, the composition comprising 5-[2-pyrazinyl]-4-methyl-1,2-3-thione or an analogue, derivative, metabolite, prodrug, solvate or pharmaceutically acceptable salt thereof.

In certain embodiments of the above mentioned aspects of the invention, the metabolite of 5-[2-pyrazinyl]-4-methyl-1,2-3-thione is the pyrrolopyrazine derivative metabolite 3 (also known as M3).

In certain embodiments, the analogue of 5-[2-pyrazinyl]-4-methyl-1,2-3-thione is anethole trithione (also known as anetol tritiona or SONICUR™).

In certain embodiments, the compound of the invention is co-administered along with, or formulated with, carboxymethyl cellulose (CMC).

In certain embodiments, the 5-[2-pyrazinyl]-4-methyl-1,2-3-thione compound is administered with, or formulated with, cysteine or an analogue, derivative, salt or solvate thereof.

In certain embodiments, the 5-[2-pyrazinyl]-4-methyl-1,2-3-thione compound is administered with, or formulated with, a chemotherapeutic agent that may be selected from the group consisting of, but not limited to, cisplatin, dexamethasone and 5-fluorouracil.

In certain embodiments, the subject has undergone cancer treatment by chemotherapy, radiotherapy or a combination thereof.

In certain embodiments, the cachexia is cancer cachexia.

### BRIEF DESCRIPTION OF THE DRAWINGS

FIG. 1 shows a Kaplan Meier's estimate of survival of mice orally administered with different doses of 5-[2-pyrazinyl]-4-methyl-1,2-3-thione prior to exposure to 10 Gy of gamma irradiation,

FIG. 2 shows a bar chart detailing the effect of various doses of 5-[2-pyrazinyl]-4-methyl-1,2-3-thione on the survival of mice exposed to 10 Gy of gamma irradiation at a dose of 1.33 Gy per minute from a ⁶⁰Co gamma irradiation source,

FIG. 3 shows a Kaplan Meier's estimate of survival of mice orally administered with different doses of N⁶-Isopentenyl Adenosine prior to exposure to 10 Gy of gamma irradiation,

FIG. 4 shows a bar chart detailing the effect of various doses of N⁶-Isopentenyl Adenosine (also known as 6-gamma-Dimethyl Allyl Amino Purine Ribose (DAPR)) on the survival of mice exposed to 10 Gy of gamma irradiation at a dose of 1.33 Gy per minute from a ⁶⁰Co gamma irradiation source,

FIG. 5 is a graph showing the 30-day survival of mice pre-treated with different doses of 5-[2-pyrazinyl]-4-methyl-1,2-3-thione and exposed to 8 Gy gamma radiation,

FIG. 6 is a graph showing the variation in body weight (as percentage of average body weight on first day of treatment) of Swiss albino mice with or without 5-[2-pyrazinyl]-4-methyl-1,2-3-thione treatment and/or exposed to gamma irradiation,

FIG. 7 is a graph showing the 30-day survival of mice with or without 5-[2-pyrazinyl]-4-methyl-1,2-3-thione treatment after exposure to different doses of gamma irradiation,

FIG. 8 shows radiation-induced micronuclei in bone marrow cells of mice, with micro-nucleated polychromatic erythrocytes being shown by arrows,

FIG. 9 shows radiation induced chromosomal aberrations in bone marrow cells of mice (a) normal metaphase showing 40 chromosomes in animals, (b) radiation-induced chromatid breaks, exchange and ring, (c) pulverisation and (d) polyploidy,

FIG. 10 shows the percentage daily weight change for each animal and the means for each treatment group for (A) groups receiving monotherapy and (B) groups receiving combination therapy with radiation. Error bars represent the SEM,

FIG. 11 shows the mean weight change as area under the curve (AUC). The AUC was calculated for the percent weight change exhibited by each animal in the study. This calculation was made using the trapezoidal rule transformation. Group means were calculated and are shown with error bars representing SEM for each group. Groups were compared using the One-Way ANOVA method. No statistically significant differences were seen between 5-[2-pyrazinyl]-4-methyl-1,2-3-thione treated and vehicle treated control groups (P=0.153),

FIG. 12 shows mean tumour volumes calculated from the length and width measurements. Error bars represent the SEM. (A) shows results for groups receiving monotherapy. (B) shows results for groups receiving combination therapy with radiation, and

FIG. 13 shows the mean weight change as area under the curve (AUG). The AUG was calculated for the tumour volume measured on each animal in the study. This calculation was made using the trapezoidal rule transformation. Group means were calculated and are shown with error bars representing SEM for each group.

### DETAILED DESCRIPTION OF THE INVENTION

Without wishing to be bound by theory, the invention is based, in part, on the inventor's unexpected finding that treatment of a subject with a composition of the invention can prevent thinning and ulceration of the mucosa of the digestive (GI) tract of a subject.

The term “mucositis” as used herein is intended to comprise alimentary mucositis. In certain embodiments, the alimentary mucositis comprises oral mucositis and/or enteritis (inflammation of the intestines, in particular the small intestine). In certain embodiments, the alimentary mucositis comprises esophagitis (inflammation of the esophagus), oropharyngeal mucositis, stomatitis (inflammation of the stomach) and/or proctitis (inflammation of the rectum).

In certain embodiments, the methods and uses of the present invention comprise administering a therapeutically effective amount of at least one of the compounds of the invention to at least one area of the digestive tract of a subject with mucositis or at risk of developing mucositis. In certain embodiments, the at least one compound may be administered to more than one area of the alimentary canal.

The term “cachexia” as used herein refers to an undesirable loss of weight by a person who is not actively trying to lose weight. The expression “reducing and/or preventing weight loss” and similar expressions as used herein include cases wherein there is no change in weight and/or wherein an increase in weight occurs.

In certain embodiments, the compositions, methods and uses extend to preventing mucositis and/or weight loss in a subject who is to undergo radiation therapy and/or chemotherapy. In certain embodiments, the subject may be administered at least one of the compounds of the present invention prior to conditioning myeloablative radiation therapy and/or chemotherapy in preparation for autologous or allogenic haematopoietic stem cell transplant.

In certain embodiments, the invention provides compositions and methods for the prophylaxis and/or treatment of mucositis and/or weight loss in a subject who has received, or who is going to receive, mucosatoxic chemotherapy with mucositis-inducing agents.

In certain embodiments, the invention provides methods and compositions for preventing and/or treating mucositis in a subject who presents with head and/or neck cancer which has been, or which is going to be, treated with radiation therapy with or without adjuvant chemotherapy.

In certain embodiments, the mucositis and/or weight loss is caused by a subject being exposed to a chemical insult, a biological insult, radiation or a combination thereof. Radiation exposure may result from radiation therapy, for example chemotherapy, radiotherapy or the like, or may result from accidental radiation exposure or exposure to radiation following a terrorist attack. The compositions, methods and uses of the present invention have further utility in relation to administration to subjects prior to, or following, space travel in order to prevent, treat or ameliorate mucositis and/or weight loss.

In certain embodiments, the methods or uses of the invention are performed prior to the subject being subjected to the insult, wherein said insult may induce or cause the progression of mucositis and/or weight loss.

In further embodiments, the methods or uses of the invention may be performed after exposure of the subject to the insult, but prior to the onset and development of mucositis and/or weight loss in the subject.

In yet further embodiments, the methods or uses of the invention may be performed on a subject after the development of mucositis and/or weight loss in the subject.

The compositions and methods of the present invention can also be used in combination with other therapies to prevent and/or treat mucositis and/or weight loss. For example, a composition comprising 5-[2-pyrazinyl]-4-methyl-1,2-3-thione and/or N⁶-isopentenyl adenosine and optionally at least one pharmaceutically acceptable carrier may be administered in combination with at least one further therapeutic agent which has a prophylactic and/or therapeutic effect on the onset or progression of mucositis, or which ameliorates at least one symptom associated with mucositis or reduces weight loss. Non-limiting examples of such further therapeutic agents include laser therapy, cryotherapy, antibiotics, cytokine-based therapies such as palifermin (brand name Kepivance, Amgen) which is a human keratinocyte growth factor (KGF), and other cytokine modulators of inflammation, such as IL-11, TGF and GM-CSF.

The compounds of the present invention can be used in the preparation of a combined medicament comprising at least one compound of the present invention along with a chemotherapeutic agent.

Chemotherapeutic agents suitable for use along with the compositions of the present invention include one or more other anti-tumour substances, for example those selected from mitotic inhibitors, such as vinblastine; alkylating agents, such as cisplatin, carboplatin, and cyclophosphamide; inhibitors of microtubule assembly, such as paclitaxel or other taxanes; anti-metabolites, such as 5-fluorouracil, capecitabine, cytosine arabinoside and hydroxyurea; intercalating antibiotics, such as, adriamycin and bleomycin; immunostimulants, such as trastuzumab; DNA synthesis inhibitors, such as, gemcitabine; enzymes, such as asparaginase; topoisomerase inhibitors, such as etoposide; biological response modifiers, such as interferon; and anti-hormones, for example, antioestrogens, such as tamoxifen, or antiandrogens, such as (4′-cyano-3-(4-fluorophenylsulphonyl)-2-hydroxy-2-methyl-3′-(trifluoromethyl)-propionanilide and other therapeutic agents and principles as described in, for example, DeVita, V. T., Jr., Hellmann, S., Rosenberg, S. A.; in: Cancer: Principles & Practice of Oncology, 5th ed., Lippincott-Raven Publishers (1997).

Methods for the administration of such a combined medicament may further be provided by the present invention. In certain embodiments, the compounds of the present invention and the chemotherapeutic agent are provided sequentially, simultaneously or separately by different routes of administration. Further, said compounds and chemotherapeutic agent may be in the same or different forms, for example a solid and a liquid. Such methods can comprise the simultaneous administration of the compounds of the present invention along with the chemotherapeutic agent. In certain embodiments, the compounds of the present invention may be administered to the subject sequentially with the chemotherapeutic agent. Where they are administered sequentially, in certain embodiments, the compounds of the present invention may be administered prior to the chemotherapeutic agent. In certain further embodiments, the compounds of the present invention may be administered following administration of the chemotherapeutic agent. In certain embodiments, the chemotherapeutic agent is provided separately to the compounds of the present invention.

In certain embodiments, the chemotherapeutic agent and compounds of the present invention are co-administered. Co-administration means that these components may be administered together as a composition, or as part of the same unitary dose. As used herein, the term “co-administration” can also mean administering the components separately, but as part of the same therapeutic regimen or treatment program. In certain embodiments, the components are administered to a subject at the same time. However, the components may also be administered separately as separate dosages or dosage forms. Where the components are administered separately, the co-administration of the components does not impose a restriction on the timing, frequency, dosage or order of administration of the components.

The structure of 5-[2-pyrazinyl]-4-methyl-1,2-3-thione (also known as oltipraz, 4-methyl-5(2-pyrazinyl)-3H-1,2-dithiole-3-thione or 5-(2-pyrazinyl)-4-methyl-l,2-dithiol-3-thione) is shown below as Formula 1.

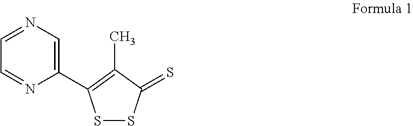

In certain embodiments, 5-[2-pyrazinyl]-4-methyl-1,2-3-thione chelates with, or forms a complex with, one or more divalent or trivalent radioactive metal ions, whereby the divalent or trivalent radioactive ions in the subject's cells or tissues are redistributed or sequestered such that the ions are limited in their capacity to participate in unwanted tissue destruction. The divalent or trivalent metal ions may be selected from, but are not limited to, the group consisting of iron, copper, nickel, calcium, magnesium, manganese, cadmium, lead, aluminium, silver, cobalt, iodine, zinc, mercury, caesium, uranium, selenium, protactinium, thorium, radium, and cerium ions or radicals.

Without wishing to be bound by theory, the inventor has identified that the therapeutic and/or prophylactic effect of 5-[2-pyrazinyl]-4-methyl-1,2-3-thione in relation to the treatment or prophylaxis of mucositis is due to enhanced expression of glutathione (GSH), glutathione reductase and/or glutathione-S-transferase.

Cytokinins are a well-known class of plant growth hormones active in promoting cell division, cell growth and differentiation and other physiological processes. Cytokinins are involved in promoting growth and cell division in explants of plant tissue in culture in standard media, which contain auxins (another class of plant hormones) as well as vitamins, mineral salts and sugar. In particular, cytokinins are active in processes regulating disease resistance, stress tolerance, drought tolerance, resistance to lodging, delayed senescence, apical dominance and assimilating partitioning in a plant (Werner et al., Proc. Natl. Acad. Sci, 98(18)10487 10492 (2001), Haberer et al., Plant Physiol., 128, pp. 354 362 (2002)).

As herein defined, the term “cytokinin” means a compound which is a plant growth substance (plant hormone) that is involved in cell growth and differentiation, as well as in other processes. In particular, the term encompasses the class of cytokinins termed “adenine cytokines”, which includes kinetin, zeatin and benzyl adenine. The term further includes “phenylurea cytokinins”, such as N,N′-diphenylurea, which although having a differing chemical composition has a similar biological activity to adenine cytokinins.

Suitable cytokinin compounds for use in the foregoing aspects of the present invention are defined below as Formula 2.

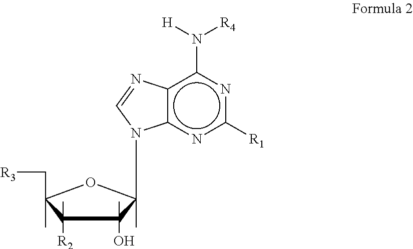

wherein:
R₁═H, R₂═CH₃, R₃═CH₃ and R₄═H, or
R₁═H or CH₃S and R₄ is as follows:

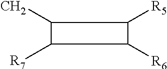

and R₅═CH₃, Cl, OH or a monophosphate group
   R₆═CH₃, CH₂OH or Cl
   R₇═H or Br, or
R₁═H and R₄ is as follows:

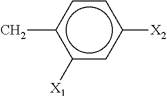

and X₁ and X₂ are independently selected from H, methyl, ethyl, hydroxyl, a halogen and carboxyl,
or R₄ is:

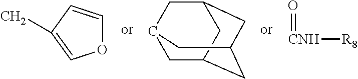

and wherein R₈ is as follows:

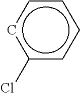

or R8 is:

(CH₂)₇CH₃
and R₂═OH and R₃═OH, monophosphate, diphosphate or triphosphate group,
or R₂ and R₃ are linked to form a 3′,5′-cyclic monophosphate derivative, or a physiologically acceptable salt of any such compound.

Formula 2 is used herein to refer to all such compounds and salts, as well as polymers of IPA, identified herein as “Poly N⁶-Isopentenyl Adenosine”, preferably comprising 2 to 3 monomers.

Listed below are chemical groups R₁ to R₄ for preferred compounds Ia to Iu of Formula 2.
Ia: ═H, R₂═OH, R₃═OH and R₄ is:

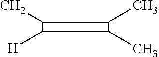

This compound is known as N⁶-(Δ²-isopentenyl) adenosine.
Ib: R₁═H, R₂═OH, R₃=monophosphate and R₄ is:

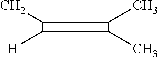

This compound is known as N⁶-(Δ²-isopentenyl) adenosine-5′-monosphosphate.
Ic: R₁═H, R₂ and R₃ are linked to form a 3′,5′-cyclic monophosphate derivative, and R₄ is:

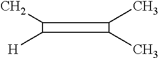

This compound is known as N⁶-(Δ²-isopentenyl) adenosine-5′-cyclic monosphosphate.
Id: R₁═H, R₂═OH, R₃═OH and R₄═CH₂C₆H₆.
This compound is known as N⁶-benzyladenosine.
Ie: R₁═H, R₂═OH, R₃=monophosphate, and R₄═CH₂C₆H₆.
This compound is known as N⁶-benzyladenosine-5′-monophosphate.
If: R₁═H, R₂ and R₃ are linked to form a 3′,5′-cyclic monophosphate derivative and R₄═CH₂C₆H₆.
This compound is known as N⁶-benzyladenosine-3′,5′ cyclic monophosphate.
Ig: R₁═H, R₂═OH, R₃═OH, and R₄ is:

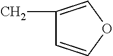

This compound is known as Furfuryladenosine.
Ih: R₁═H, R₂═OH, R₃=monophosphate and R₄ is:

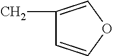

This compound is known as N⁶-furfuryladenosine-5′ monophosphate.
Ii: R₁═H, R₂ and R₃ are linked to form a 3′,5′-cyclic monophosphate derivative and R₄ is:

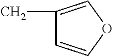

This compound is known as N⁶-furfuryladenosine-3′,5′-cyclic monophosphate.
Ij: R₁═H, R₂═OH, R₃═OH and R₄ is:

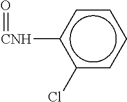

This compound is known as N-(purin-6-ylcarbamoyl)-o-chloroaniline ribonucleoside.
Ik: R₁═H, R₂═OH, R₃=monophosphate and R₄ is:

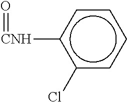

This compound is known as N-(purin-6-ylcarbamoyl)-o-chloroaniline ribonucleoside-5′ monophosphate.
Il: R₁═H, R₂═OH, R₃═OH and R₄ is:

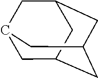

This compound is known as N⁶-adamantyladenosine.
Im: R₁═H, R₂═OH, R₃=monophosphate and R₄ is:

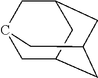

This compound is known as N⁶-adamantyladenosine-5′-monophosphate.
In: R₁═H, R₂═OH, R₃═OH and R₄ is:

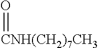

This compound is known as N-(purin-6-ylcarbamoyl)-n-octylamine ribonucleoside.
Io: R₁═H, R₂═OH, R₃=monophosphate and R₄ is:

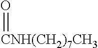

This compound is known as N-(purin-6-ylcarbamoyl)-n-octylamine ribonucleoside-5′-monophosphate.
Ip: R₁═H, R₂ and R₃ are linked to form a 3′,5′-cyclic monophosphate derivative and R₄ is:

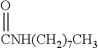

This compound is known as N-(purin-6-ylcarbamoyl)-n-octylamine ribonucleoside-3′,5′-cyclic monophosphate.
Iq: R₁═CH₃S, R₂═OH, R₃═OH and R₄ is:

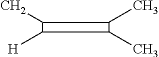

This compound is known as N6-(Δ2-isopentyl)-2-methylioadenosine.
Ir: R₁═H, R₂═OH, R₃═OH and R₄ is:

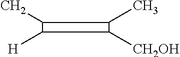

This compound is known as N⁶-(4-hydroxy-3-methyl-trans-2-butenyl)-adenosine.
Is: R₁═H, R₂═OH, R₃═OH and R₄ is:

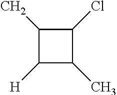

This compound is known as N⁶-(3-chloro-trans-butenyl) adenosine.
It: R₁═H, R₂═OH, R₃═OH and R₄ is:

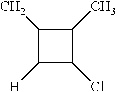

This compound is known as N⁶-(3-chloro-cis-2-butenyl) adenosine.
Iu: R₁═H, R₂═CH₃, R₃═CH₃ and R₄═H.

The present invention further extends to one or more metabolites of the compounds of Formula 2. For example, preferred metabolites include N⁶-(Δ²-isopentenyl) adenine, 6-N-(3-methyl-3-hydroxybutylamino) purine, adenine, hypoxanthine, uric acid and methylated xanthines.

Without wishing to be bound by theory, it is hypothesised that the cytokinin compound of Formula 2 enhances the cellular production of phase II detoxification enzymes following their depletion by radiation exposure. The phase II detoxification enzymes may be selected from the group consisting of glutathione S transferase, gamma-glutamylcysteine synthetase, glutathione reductase, glutathione peroxidase, epoxide hydrase, AFB-1 aldehyde reductase, glucuronyl reductase; glucose-6-phosphate dehydrogenase, UDP-glucuronyl transferase and AND(P)H:quinone oxidoreductase.

In certain aspects, the present invention further extends to methods, uses and compositions of the invention comprising at least one of the following compounds:

ADT, having the general structure:

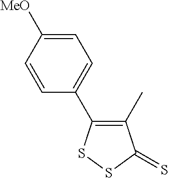

ADO, having the general structure:

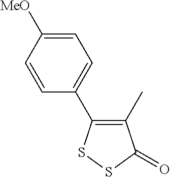

1,2-Dithiole 3-thione having the structure:

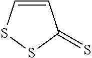

Lipoamide (1,2-dithiolane), having the structure:

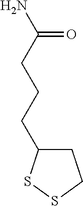 [SMILES: NC(=O)CCCC1CCSS1]

1,3-dithiole 2-thione having the structure:

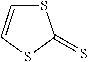

[1,2]Dithiolo[4,3-c]-1,2-dithiole-3,6-dithione having the structure:

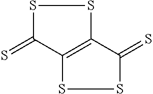

In certain aspects, the present invention further extends to methods, uses and compositions of the invention comprising at least one of the following compounds:

1,2-Dithiolane class 1 compounds having the general structure:

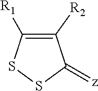

1,2-Dithiole class 2 compounds having the general structure:

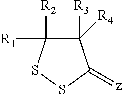

1,3-Dithiole class 3 compounds having the general structure:

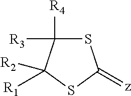

1,3-Dithioloane class 4 compounds having the general structure:

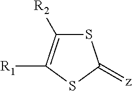

wherein Z═S, O, NR, R₂, CR₂ and Z can have the designations optionally and independently for all the classes. R in this case includes H, alkyl (C1-C5), alkoxy (C1-C5), alkoxycarbononyl (C1-C5). R₂ can form spiro rings about the ring carbon atom.

For the thiolane classes, the ring carbon atoms can be doubly substituted. R₁-R₄ are the main ring substituents for all classes and, in order to cover a wide variety of substituents, should include optionally and independently H, alkyl, aryl, heterocyclic, halogen, alkoxycarbonyl (C1-C5) or carboxyl.

R₁, R₂, R₃ and R₄ can form a Spiro ring around the carbon atom to which they are attached or they can form fused or bridged rings to adjacent carbons atoms.

The following definitions cover the majority of the compounds as described herein.

An alkyl is defined herein as a C1-C10 linear or branched chain, saturated or unsaturated, which can optionally be singly or multiply substituted by halogen, alkyl (C1-C5), hydroxyl, alkoxy (C1-C5), alkoxycarbonyl, (C1-C5), carboxyl, amido, alkyl amido (C1-C5), amino, mono and dialkyl amino (C1-C5), alkyl carbamoyl (C1-C5), thiol, alkylhio (C1-C5) or benzenoid aryl.

An aryl is defined herein as any optionally singly or multiply substituted benzenoid group (C6-C14). The substituents are defined below.

Heterocyclic radical means any 4, 5 or 6 membered, optionally substituted heterocyclic ring, saturated or unsaturated, containing 1-3 ring atoms of N, O or S, the remaining atoms being carbon.

Substituents on the aryl or heterocyclic radical include halogen, alkyl (C1-05), hydroxyl, alkoxy (C1-C5), alkoxycarbonyl, (C1-C5), carboxyl, amido, alkyl amido (C1-C5), amino, mono and dialkyl amino (C1-C5), alkyl carbamoyl (C1-C5), thiol, alkyl thio (C1-C5) or benzenoid aryl, cyano, nitro, halo alkyls, alklsulfonyl (C1-C5), sulfonate. Two of such substituents can be part of a fused ring, which can be either saturated, or unsaturated, heterocyclic or carbo cyclic.

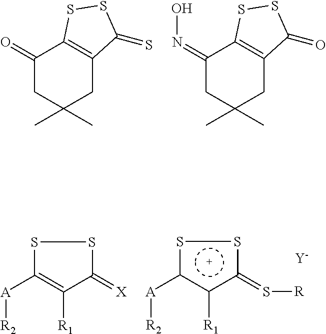

in which:
X is chosen from:
   ═S
   ═O
   ═N—OH
   ═N—R₅
      R₅, being a C₁-C₆ alkyl or an aryl group,
   ═N—NH—CO—NH₂
   ═N—NH—CS—NH₂, and

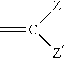

Z and Z′ being electron-attracting groups such as ester or cyano groups.
A is chosen from a >C═N—OH group or a group of formula >C═N—OR₃ (where R₃ is chosen from hydroxyl, amino, chloro and C1-C4, alkoxy groups, an aryl (C1-C6 alkyl) group, a (C1-C6 alkyl) carbonyl group and an aryl (C1-C6 alkyl) carbonyl group).
A may also be chosen from a >C═O group, a >C═N—R₄ group, R₄ being a C1-C6 alkyl group or an aryl group, and a CHOH group.
R₁ and R₂ are chosen, independently of one another, from hydrogen, a halogen, a nitro group, a nitroso group, a thiocyano group, a C1-C6 alkyl group, a C2-C6 alkenyl group, an aryl group, aryl (C1-C6 alkyl) group, an aryl (C2-C6 alkenyl) group, a carboxyl group, a (C1-C6 alkyl) carbonyl group, an arylcarbonyl group, a (C1-C6 alkoxy) carbonyl group, a (C1-C6 alkoxy) carbonyl (C1-C6 alkyl) group, a C1-C6 alkoxy group, a trifluoromethyl group, an amino group, a di (C1-C6 alkyl) amino (C1-C6 alkyl), an acylamino group of formula —NHCOCₙH₂ₙ₊₁ with n from 0 to 6, a group —NH—CSCₙH₂ₙ₊₁ with n from 0 to 6, a terpenyl group, a cyano group, a C2-C6 alkynyl group, a C2-C6 alkynyl group substituted with a C1-C6 alkyl or an aryl group, a hydroxy (C1-C6 alkyl) group, a (C1-C6 acyl) oxy (C1-C6 alkyl) group, a (C1-C6 alkyl) thio group and an arylthio group; or
alternatively R₁ and R₂ together form a mono- or polycyclic C2-C20 alkylene group optionally comprising one or more hetero atoms, with the exception of the 2,2dimethyltrimethylene group, or a C3-C12 cycloalkylene group.
R is chosen from a C1-C6, alkyl group, and their pharmaceutically acceptable salts.

In the foregoing definition, aryl group or aryl fraction of an arylalkyl group denotes an aromatic carbon-based group such as a phenyl or naphthyl group or an aromatic heterocyclic group such as a thienyl of furyl group, it being possible for these groups to bear one or more substituents chosen from a halogen atom, a C1-C4 alkyl group, a C1-C4 alkoxy group, a trifluoromethyl group, a nitro group and a hydroxyl group,
oximes of 1,2-dithiole-3-thione derivatives such as shown below,

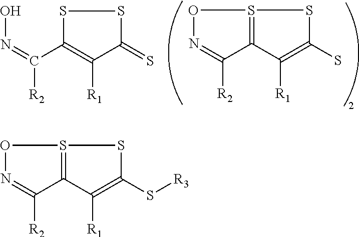

additionally Aldehydes or Ketones of previously identified compounds, such as shown below,

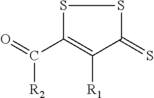

one or more of the following compounds wherein A is a group C═N═OR′₃ where R′₃ is an optionally substituted C1-C6 alkyl group, in particular substituted with one or more groups chosen from hydroxyl, amino, chloro, bromo, fluoro, iodo and C1-C4 alkoxy groups, or an aryl (C1-C6 alkyl) group, that is to say compounds of formula

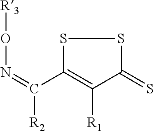

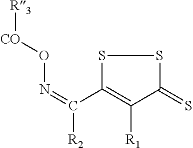

in which R₃ has the meaning given above,
   one or more of the following compounds in which A is a group C═N—O—CO—R″₃, R″₃ being chosen from a hydrogen atom, an optionally substituted C1-C6 alkyl group, an aryl group and an aryl (C1-C6 alkyl) group, that is to say compounds of formula

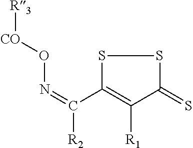

in which R″₃ is chosen from a hydrogen atom, an optionally substituted C1-C6 alkyl group and an aryl group.

Another group of compounds is formed in which A is a CH—OH group, that is to say the compounds of formula:

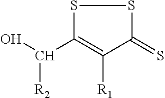

Another group of compounds is formed by compounds in which A is a group comprising C═N—R, wherein R is a C1-C6 alkyl or an aryl group, that is to say compounds of formula

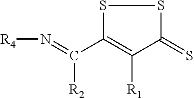

Another group of compounds includes compounds in which A is a C═O group and X is an oxygen atom, that is to say compounds of formula:

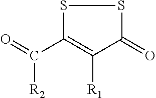

in which
R₁ is chosen from hydrogen, a halogen, a nitro group, a nitroso group, a thiocyano group, a C1-C6 alkyl group, a C2-C6 alkenyl group, an aryl group, an aryl (C1-C6 alkyl) group, an aryl (C2-C6 alkenyl) group, a carboxyl group, a (C1-C6 alkyl) carbonyl group, an arylcarbonyl group, a (C1-C6 alkoxy) carbonyl group, a (C1-C6 alkoxy) carbonyl (C1-C6 alkyl) group a (C1-C6 alkoxy group, a trifluoromethyl group, an amino group, a di (C1-C6 alkyl) amino (C1-C6 alkyl) group, an acylamino group of formula —NHCOCₙH₂ₙ₊₁ with n from 0 to 6, a group —NH—CSCₙH₂ₙ₊₁ with n from 0 to 6, a terpenyl group, a cyano group, a C1-C6 alkynyl group, a C2-C6 alkynyl group substituted with a C1-C6 alkyl or an aryl group, a hydroxy (C1-C6 alkyl) group, a (C1-C6 acyl)-oxy(C1-C6 alkyl) group, a C1-C6 alkyl)thio group and an arylthio group.
R₂ is chosen from a nitro group, a nitroso group, a thiocyano group, a C1-C6 alkyl group, a C2-C6 alkenyl group, an aryl group, an aryl (C1-C6 alkyl) group, an aryl (C1-C6 alkenyl) group, a carboxyl group, a(C1-C6 alkyl)carbonyl group, an arylcarbonyl group, a (C1-C6 alkoxy)carbonyl group, a (C1-C6 alkyl) group, a trifluoromethyl group, a di(C1-C6 alkyl)amino(C1-C6 alkyl) group, an acylamino group of formula —NHCOCₙH₂ₙ₊₁ with n from 0 to 6, a group —NH—CSCₙH₂ₙ₊₁ with n from 0 to 6, a terpenyl group, a cyano group, a C2-C6 alkynl group, a C2-C6 alkynyl group substituted with a C1-C6 alkyl or an aryl group, a hydroxy (C1-C6 alkyl) group, a C1-C6 acyl-oxy(C1-C6 alkyl) group, a (C1-C6 alkyl)thio group and an arylthio group; or
   alternatively, R₁ and R₂ together form a mono- or polycyclic C2-C20 alkylene group optionally comprising one or more hetero atoms,

A further group of compounds includes one or more of the following compounds as shown below:

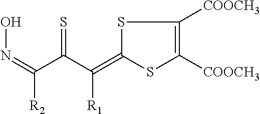

R₁ and R₂ are chosen, independently of one another, from hydrogen, a halogen, a nitro group, a nitroso group, a thiocyano group, a C1-C6 alkyl group, a C2-C6 alkenyl group, an aryl group, aryl(C1-C6 alkyl) group, an aryl (C2-C6 alkenyl) group, a carboxyl group, a (C1-C6 alkyl)carbonyl group, an arylcarbonyl group, a (C1-C6 alkoxy)carbonyl group, a (C1-C6 alkoxy)carbonyl (C1-C6 alkyl) group, a C1-C6 alkoxy group, a trifluoromethyl group, a di(C1-C6 alkyl)amino(C1-C6 alkyl) group, an acylamino group of formula —NHCOCₙH₂ₙ₊₁ with n from 0 to 6, a group —NH—CSCₙH₂ₙ₊₁ with n from 0 to 6, a terpenyl group, a cyano group, a C2-C6 alkynyl group, a C2-C6 alkynyl group substituted with a C1-C6 alkyl or an aryl group, a hydroxy(C1-C6 alkyl) group, a (C1-C6 acyl) oxy (C1-C6 alkyl) group, a (C1-C6 alkyl) thio group and an arylthio group;
or alternatively R₁ and R₂ together form a mono- or polycyclic C2-C20 alkylene group optionally comprising one or more hetero atoms.

R is chosen from a C1-C6 alkyl group, and their pharmaceutically acceptable salts.

In the foregoing definition, aryl group or aryl fraction of an arylalkyl group denotes an aromatic carbon-based group such as a phenyl or naphthyl group or an aromatic heterocyclic group such as a thienyl of furyl group, it being possible for these groups to bear one or more substituents chosen from halogen atom, a C1-C4 alkyl group, a C1-C4 alkoxy group, a trifluoromethyl group, a nitro group and a hydroxyl group, one more of the following isobenzothiazolone derivative having the structure:

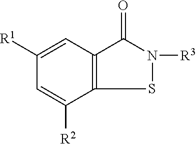

In this structure at least one of R¹ and R² is preferably nitro, arylazo, substituted arylazo, benzylideneamino or substituted benzylideneamino. When only one of R¹ and R² is so substituted, one of R¹ and R² may be hydrogen. The R³ substituent is selected from alkyl groups in less than about 7 carbon atoms, amino, hydroxyl, alkoxyl, and aryl groups (and functionalized forms thereof).

Preferred species of the isobenzothiazole derivative of the present invention comprise R¹ as nitro or arylazo and R² as hydrogen, for example. Examples include compounds where R² is hydrogen and R¹ is phenylazo; substituted arylazo such as 4-hydroxyphenylazo; 4 nitro-2-methylphenylazo; 2-hydroxy-1-napthylazo; 2-hyroxy-5-methylphenylazo; 2-hydroxy-4-methyl-5-nitrophenylazo; 4-hydroxy-1-napthylazo; 4-hydroxy-3-methyl-1-napthylazo; 4-hydroxy-5-aza-1-napthylazo; 2 amino-1-napthylazo; 1-hydroxy-2-napthylazo; 3-N,Ndimethylaminopropylcarboxyamido-1-hydroxy-4-naphthylazo; 1-hydroxy-4-methoxy-2-naphthylazo, 2-hydroxy-3-carboxy-1-naphthylazo; 1-hydroxy-3,6-disulfonato-2-naphthylazo; 2,3-dihydroxy-1-naphthylazo; or 2-hydroxy-3,5-dimenthyl-1-phenylazo. In one particular embodiment R¹ is the substituted benzylideneamino, 2,4-dinitrobenzylideneamino and R² is hydrogen. Additionally R¹ is hydrogen and R² is 2-hydroxy-1-naphthylazo or 4-hydroxy-Iphenylazo.

In one aspect, R³ substituents with sufficient polarity to confer aqueous solubility upon the compound. For example, R³ may be —(CH₂)ₙR⁴R⁵ where n is from 2 to 6 and R⁴ and R⁵ are simple alkyls or hydrogens. Other possible water solubilizing side chains include 3-carboxypropyl, sulfonatoethyl and polyethyl ethers of the type —CH₂(CH₂OCH₂)CH₃ where n is less than 10. Preferred compounds include R³ side chains containing aminoalkyl, carboxyalkyl, omega amino polyethyl ethers and N-haloacetyl derivatives. In a broader sense, for various utilities R³ may be alkyl, aryl, heteroaryl, alkoxy, hydroxyl or amino groups. When including substitutions for solubility or reactivity purposes, R³ may be aminoalkyl, carboxyalkyl, hydroxyalkyl or haloalkyl. The aryl or heteroarl R³ moieties may be substituted, for example as aminoaryl, carboxyaryl or hydroxyaryl. Also included are one or more of the following Isobenzothiazolone derivatives having the structure:

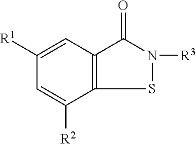

wherein at least one of R¹ and R² is nitro, arylazo, substituted arylazo, benzylideneamino or substituted benzylideneamino and one of R¹ and R² may be hydrogen and R³ is a aminoallayl, aminoaryl and aminoheteroaryl, carboxyalkyl, carboxyaryl or carboxyheteroaryl covalently linked to a polymer comprising amino or hydroxy groups. The spacer arm R³ can comprise oligomers or polyethylene-glycol and its derivatives. In one aspect, R³ may be 17-chloracetamido-3,6,9,12,15-pentaoxyheptadecyl where hexaethylene glycol has been chloroacetamidated. When the polymer groups Y¹ and R³ comprise carboxyl groups, the covalent linkage is preferably through an ester bond. When the polymer comprises amino groups, the analogue covalent linkage is through an amide bond. The amine bearing polymer, when coupled to R³, may be a polymer such as chitosan, polyalkylamine, aminodextran, polyethyleneimine, polylysine or amitryrene.
The R³ substituents of the present invention may also comprise an alkyl linked to an amine bearing polymer by amine displacement of a halogen from an alpha-haloalkyl or alpha-haloalkylcarbox amido R³ precursor. In the case of aminoalkyl or aminoaryl groups the R³ substituent may also be covalently linked to a polymer such as polyepichlorohydrin, chloromethylpolystyrene, polyvinylalochol or polyvinylpyridine. The R³ substituent of the present invention may generally be an aminoalkyl, hydroxyalkyl, aminoaryl or hydroxyaryl group linked to a polymer comprising carboxyl groups through amide or ester linkages.
When polymers are involved in the R³ structure, the polymer may be one such as polyacrylic acid, polymethacrylic acid, polyilaconic acid, oxidized polyethylene oxide, poly(methylmethacrylate/methacrylic acid), carboxylnethyl cellulose, carboxymethyl agarose or carboxymethyl dextran. When such a carboxyl polymer is involved, the R³ may be aminoalkyl (such as 8 aminohexyl, for example), hydroxyalkyl, aminoaryl or hydroxyaryl linked to the polymer through amide or ester linkages. In such cases, an R³ precursor function may bear an amine or hydroxyl group to be covalently linked to a polymer by reaction with an acid anhydride-bearing polymer or by coupling with a carboxylate bearing polymer through carbodimideinduced bond formation.
The R³ substituent or precursor thereto in the compounds of the present invention may also be a haloalkyl or carboxylialoalkyl moiety such as chloracetamido. Such a substituent may be readily coupled to an amine bearing polymer by amine displacement of the halogen.

“Aryl,” as used herein, is intended to include organic residues derived from aromatic hydrocarbon or aromatic heterocyclic ring systems. Accordingly aryl groups include the unsubstituted ring residues such as phenyl and naphthyl and substituted forms thereof. Heterocyclic or heteroaryl residues may be those comprising one or more heteroatoms (e.g., nitrogen, oxygen, sulphur) in the ring system such as pyridyl, oxazolyl, quinoly), thiazolyl and substituted forms thereof.

“Alkyl” as used herein, is intended to include aliphatic and cyclic organic residues having a carbon at a point of attachment. Accordingly, alkyl groups include unsubstituted hydrocarbon residues of the formula CₙH₂ₙ₊₁ and substituted and cyclic forms thereof. Such hydrocarbons are usually of the lower alkyl class which have six carbons or less. It is understood that larger alkyl groups may be used. Alkyl includes substituted residues which are intended to include the hydrocarbon residues bearing one or more, same or different, functional groups as described below.

The alkyl and aryl group previously described may be substituted with functional groups. Such functional groups include essentially all chemical groups which can be introduced synthetically and result in stable compounds. Examples of these functional groups are hydroxyl, halogen (fluoro, chloro, bromo), amino (including alkylamino and dialkylamino), cyano, nitro, carboxy (including carbalkoxy), carbamoyl (including N and N,N alkyl), sulfb, alkoxy, alkyl, aryl, and arylazo, one or more of the following compounds

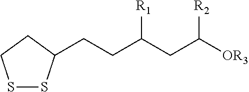

wherein R₁ and R₂ are independently (═O) or —OR, where R is H or (C1-C4) alkyl; and R₃ is H or (C1-C4) alkyl. Preferably, R₃ is H. Preferably R¹ and R₂ are (═O) or OH.

Also included is one or more of the following compounds

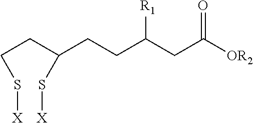

wherein X is H or both X's represent a direct bond between the two sulphur atoms; R₁ is (═O) or —OH; and R₂ is H, Na, K or (C1-C4) alkyl. In particular, the compound may be 3-keto lipoic acid, 3-hydroxy lipoic acid, 3-keto dihydrolipoic acid or 3-hydroxy dihydrolipoic acid.
1,2-dithiol-3 thione derivative of a formula shown below:

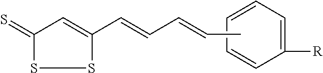

wherein R denotes hydrogen, halogen, lower alkoxy group, lower alkyl group, amino group, lower alkylsubstituted amino group or lower alkoxycarbonyl group. The term “lower” as used herein means methyl, ethyl, propyl and butyl, as well as its structural isomers such as isopropyl, isobutyl and tertiary butyl.

Among the compounds of the formula shown above, preferred compounds include:
5-(4-phenyl-1,3-butadienyl)-1,2-dithiol-3-thione,
5-4(4-chlorophenyl)-1,3-butadienyl-1,2-dithiol-3-thione,
5-{4(4-methoxyphenyl)-1,3-butadienyl}-1,2-dithiol-3-thione,
5-{4-(p-toluoyl)-1,3-butadienyl}-1,2-dithiol-3-thione,
5-{4-(o-chlorophenyl)-1,3-butadienyl}-1,2-dithiol-3-thione, and
5-{4-(m-(methylphenyl)-1,3-butadienyl}-1,2-ffithiol-3-thione.

The following compounds are also included:

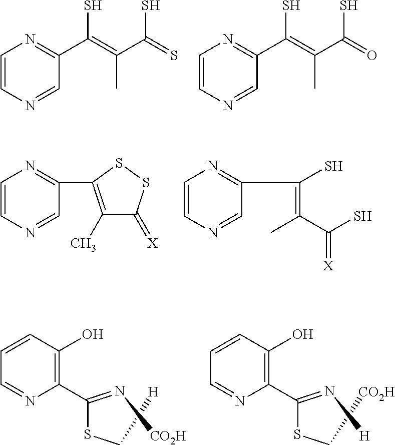

and 1,2-dithiole of the formula:

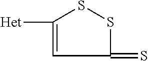

wherein Het represents pyrimidin-2-yl, pyrimidin-4-yl, or pyrimidin-5-yl, or a said pyrimidin-2-yl, pyrimidin4-yl or pyrimidin-5-yl substituted by halogen, alkyl of 1 through 4 carbon atoms, alkoxy of 1 through 4 carbon atoms, mecapto, alkylthio of 1 through 4 carbon atoms, or dialkylamino having 1 through 4 carbon atoms in each alkyl, and R represents halogen, alkyl of 1 through 4 carbon atoms, alkyl of 1 through 4 carbon atoms substituted by alkoxycarbonyl having 1 through 4 carbon atoms in the alkoxy, carboxy, alkoxycarbonyl having 1 through 4 carbon atoms in the alkoxy, carbamoyl, N-alkylcarbamoyl having 1 through 4 carbon atoms in the alkyl, or R—CH(OH)— in which R represents hydrogen or alkyl of 1 through 3 carbon atoms.

Examples of N⁶ benzyl adenosine or an analogue, derivative, metabolite, prodrug or pharmaceutically acceptable salt thereof are described below.

In certain further embodiments, the N⁶ benzyl adenosine is N⁶-Benzyl-adenosine-5′ monophosphate, which is shown below as a compound having Formula 3. This compound has a molecular weight of 437.215 and a molecular formula of C₁₇H₂₀N₅O₇P.

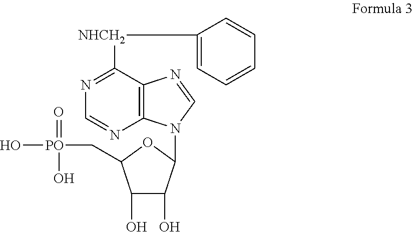

In certain further embodiments, the N⁶ benzyl adenosine is (N⁶-Benzyl)Adenyl-p-(N⁶-Benzyl)Adenyl-p-(N6-Benzyl) Adenosine, which is shown below as a compound having Formula 4. This compound has a molecular weight of 1373.39.

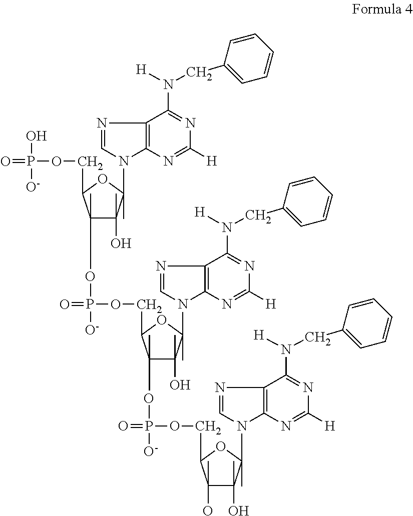

Carboxymethyl cellulose (CMC) is a cellulose derivative with carboxymethyl groups bound to some of the hydroxyl groups of the glucopyranose monomers that make up the cellulose backbone. The active compounds disclosed herein can, as noted above, be prepared in the form of their pharmaceutically acceptable salts. Pharmaceutically acceptable salts are salts that retain the desired biological activity of the parent compound and do not impart undesired toxicological effects. Examples of pharmaceutically acceptable salts are discussed in Berge et al., 1977, “Pharmaceutically Acceptable Salts,” J. Pharm. ScL, Vol. 66, pp. 1-19.

The active compounds disclosed may also be prepared in the form of their solvates. The term “solvate” is used herein in the conventional sense to refer to a complex of solute (e.g., active compound or salt of active compound) and solvent. If the solvent is water, the solvate may be conveniently referred to as a hydrate, for example, a hemihydrate, monohydrate, dihydrate, trihydrate, tetrahydrate and the like.

The invention further extends to prodrugs of the compounds of the present invention which can convert to the biologically active compound by metabolism or hydrolysis. A prodrug of any of the compounds can be made using pharmacological techniques known to those skilled in the art.

Metabolites may result from the metabolism, for example by molecular rearrangement, or hydrolysis of the compounds of the invention following administration to a subject.

The present invention is further intended to encompass, in addition to the use of the above listed compounds, the use of homologues and analogues of such compounds. In this context, homologues are molecules having substantial structural similarities to the above-described compounds and analogues are molecules having substantial biological similarities regardless of structural similarities.

The invention further provides kits for carrying out the therapeutic regimens of the invention. Such kits may comprise, in one or more containers, therapeutically effective amounts of the compositions of the invention in a pharmaceutically acceptable form. Such kits may further include instructions for the use of the compositions of the invention or the performance of the methods of the invention, or may provide further information to provide a physician with information appropriate to treating mucositis.

As used herein, the term “subject” refers to an animal, preferably a mammal, and in particular a human. In certain embodiments the subject is a mammal, in particular a human, who has been, or who is going to be, exposed to radiation, for example radiation therapy such as chemotherapy or radiotherapy.

Suitably the composition of the invention is administrated by parenteral administration. Parenteral administration may be intravenous administration or subcutaneous administration. In further embodiments the route of administration is rectal, for example by means of a suppository, transdermal or transmucosal.

In certain embodiments the composition for the treatment and/or prophylaxis of mucositis, weight loss and/or cachexia may be administered by topical application including, but not limited to, buccal and sublingual administration. Suitable formulations for topical administration include creams, gels, jellies, mucliages, pastes and ointments. In certain embodiments, the composition may be formulated for transdermal administration, for example in the form of transdermal patches.

The effective amount of the composition for the treatment and/or prophylaxis of mucositis and/or weight loss may be provided in a single dosage regimen or a multi-dose regimen.

In certain embodiments the composition may be administered orally, for example in the form of an oral rinse, or is administered to the lungs as an aerosol via oral or nasal inhalation. For administration via the oral or nasal inhalation routes, preferably the active ingredient will be in a suitable pharmaceutical formulation and may be delivered using a mechanical form including, but not restricted to, an inhaler or nebuliser device.

For intravenous injection, the active ingredient will be in the form of a parenterally acceptable aqueous solution which is pyrogen-free and has suitable pH, isotonicity and stability. Methods of preparation of suitable solutions using, for example, isotonic vehicles such as sodium chloride injection, Ringer's injection or Lactated Ringer's injection will be known to those of relevant skill in the art. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

Various delivery systems are known and can be used to administer the compositions of the present invention. More specifically, the compositions may be administered via microspheres, liposomes, or other microparticulate delivery systems or sustained release formulations placed in certain tissues including blood. Suitable examples of sustained release carriers include semipermeable polymer matrices in the form of shared articles, for example suppositories or microcapsules. Implantable or microcapsular sustained release matrices such as polylactides are also provided.

Examples of the techniques and protocols mentioned above and other techniques and protocols which may be used in accordance with the invention can be found in Remington's Pharmaceutical Sciences, 18th edition, Gennaro, A. R., Lippincott Williams & Wilkins; 20th edition (Dec. 15, 2000) ISBN 0-912734-04-3 and Pharmaceutical Dosage Forms and Drug Delivery Systems; Ansel, H. C. et al. 7th Edition ISBN 0-683305-72-7, the entire disclosures of which are herein incorporated by reference.

The composition of the invention is preferably administered to an individual in a “therapeutically effective amount” as defined hereinafter. The actual amount administered in order to achieve these effects, as well as the rate and time-course of administration, will depend on, and can be determined with due reference to, the nature and severity of the condition which is being treated, as well as factors such as the age, sex, weight of the patient to be treated and the route of administration. Toxicity and efficacy of the compositions can be determined by standard pharmaceutical procedures.

Unless otherwise defined, all technical and scientific terms used herein have the meaning commonly understood by a person who is skilled in the art in the field of the present invention.

The compounds disclosed herein extend to “other forms” of said compounds, said other forms including the well known ionic, salt, solvate, and protected forms of these substituents. For example, a reference to carboxylic acid (—COOH) also includes the anionic (carboxylate) form (—COO), a salt or solvate thereof, as well as conventional protected forms. Similarly, a reference to an amino group includes conventional protected forms of an amino group. Similarly, a reference to a hydroxyl group also includes the anionic form (—O—), a salt or solvate thereof, as well as conventional protected forms.

Certain compounds may exist in one or more particular geometric, optical, enantiomeric, diasteriomeric, epimeric, atropic, stereoisomeric, tautomeric, conformational or anomeric forms, including, but not limited to cis- and trans-forms, E- and Z-forms, c-, t- and r-forms, endo and exo-forms, R—, S— and meso forms, D- and L-forms, d- and l-forms, (+) and (−) forms, keto-, enol- and enolate-forms, syn and anti-forms, synclinal and anticlinal forms, alpha and beta forms, axial and equatorial forms, boat-, chair-, twist-, envelope-, and halfchair-forms, and combinations thereof, herein collectively referred to as “isomers” or “isomeric forms”.

Unless otherwise specified, a reference to a particular compound includes all such isomeric forms, including (wholly or partial) racemic and other mixtures thereof. Methods for the preparation (e.g. asymmetric synthesis) and separation (e.g. fractional crystallisation and chromatographic means) of such isomeric forms are either known in the art or are readily obtainable. Unless otherwise specified, a reference to a particular compound also includes ionic, salt, solvate and protected forms thereof.

The phrase “substituted” or “optionally substituted” as used herein means a parent group which may be unsubstituted or which may be substituted.

Unless otherwise specified, the term “substituted” as used herein relates to a parent group which bears one or more substituents. The term “substituent” is used herein in the conventional sense and refers to a chemical moiety which is attached to, or if appropriate, fused to, a parent group. A wide variety of substituents are well known, and methods for their formation and introduction into a variety of parent groups are also well known to the person skilled in the art.

Throughout the specification, unless the context demands otherwise, the terms “comprise” or “include”, or variations such as “comprises” or “comprising”, “includes” or “including” will be understood to imply the inclusion of a stated integer or group of integers, but not the exclusion of any other integer or group of integers.

As used herein, terms such as “a”, “an” and “the” include singular and plural referents unless the context clearly demands otherwise. Thus, for example, reference to “an active agent” or “a pharmacologically active agent” includes a single active agent as well a two or more different active agents in combination, while references to “a carrier” includes mixtures of two or more carriers as well as a single carrier, and the like.

As used herein, the term “therapeutically effective amount” means the amount of a composition which is sufficient to show benefit to the subject. In particular, the benefit may be the treatment, partial treatment or amelioration of at least one symptom associated with mucositis. In the case of prophylaxis of mucositis, the term “therapeutically effective amount” relates to the amount of a composition which is required to prevent or suppress the initial onset, progression or recurrence of mucositis, or at least one symptom thereof.

As used herein, the term “treatment” and associated terms such as “treat” and “treating” mean the reduction of the progression, severity and/or duration of mucositis, the amelioration of at least one symptom thereof or the reduction or prevention of weight loss/cachexia. The term “treatment” therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery or that no weight loss or cachexia occurs. Similarly, “prophylactic” does not necessarily mean that the subject will not eventually contract mucositis or cachexia or undergo weight loss. Accordingly, therapeutic and prophylactic treatments include amelioration of the symptoms of mucositis and preventing or otherwise reducing the risk of developing mucositis, cachexia and/or weight loss. In this context, the term “prophylactic” may be considered as reducing the severity or the onset of mucositis, cachexia and/or weight loss and the term “therapeutic” may be considered as reducing the severity of existing mucositis, cachexia and/or weight loss.

The invention will now be described with reference to the following examples which are provided for the purpose of illustration and are not intended to be construed as being limiting on the present invention, and further, with reference to the figures.

### EXAMPLES

### Example 1

### Evaluation of radioprotective efficacy of 5-[2-pyrazinyl]-4-methyl-1,2-3-thione (oltipraz)

This experiment evaluated the efficacy and safety of 5-[2-pyrazinyl]-4-methyl-1,2-3-thione (oltipraz) as a radioprotective agent.

### Acute Toxicity Studies

The animals were allowed to fast for 18 hours and administered with 0, 100, 200, 400, 500, 600, 700, 800, 1000, 1250, 1500, 1750 and 2000 mg/kg of 5-[2-pyrazinyl]-4-methyl-1,2-3-thione and observed for 14 days post-drug treatment.

### Treatment Group 1—CMC and Irradiation

Animals of this group received 0.5% carboxymethyl cellulose (CMC) orally before exposure to 10 Gy gamma irradiation.

### Treatment Group 2—5-[2-Pyrazinyl]-4-Methyl-1,2-3-Thione and Irradiation

Animals of this group were treated with 5, 10, 25, 50, 100, 150, 200 or 250 mg/kg body weight of 5-[2-pyrazinyl]-4-methyl-1,2-3-thione orally once before exposure to 10 Gy of gamma radiation.

### Irradiation

One hour after the administration of CMC or 5-[2-pyrazinyl]-4-methyl-1,2-3-thione, the prostrate and immobilized animals, achieved by inserting cotton plugs in the restrainer, were whole-body exposed to ⁶⁰Co gamma radiation (Theratron, Atomic Energy Agency, Canada) in a specially designed well-ventilated acrylic box. A batch of ten animals was irradiated each time at a dose rate of 1.33 Gy/min.

### Results

### Acute Toxicity Studies

The animals receiving different doses of 5-[2-pyrazinyl]-4-methyl-1,2-3-thione did not show any signs of toxicity up 2 g/kg and not a single mortality was observed up to 14 days. Therefore 5-[2-pyrazinyl]-4-methyl-1,2-3-thione up to 2 g was considered completely safe for administration. Higher doses could not be evaluated owing to problems in drug dissolution.

The radioprotective efficacy of 5-[2-pyrazinyl]-4-methyl-1,2-3-thione was evaluated by treating mice with 0, 5, 25, 50, 100, 150, 200 and 250 mg/kg body weight 5-[2-pyrazinyl]-4-methyl-1,2-3-thione before whole-body exposure to 10 Gy gamma radiation. After irradiation, the animals were monitored daily for 30 days for the development of symptoms of radiation sickness and mortality.

Exposure of the CMC and irradiation group to 10 Gy induced symptoms of severe radiation sickness, such as a reduction in food and water intake, irritability, lethargy, body weight loss, diarrhoea, lacrimation, facial edema, emaciation and epilation. The first mortality in the CMC and irradiation group was observed at day 4 and all of the irradiated animals died by day 18 post-irradiation.

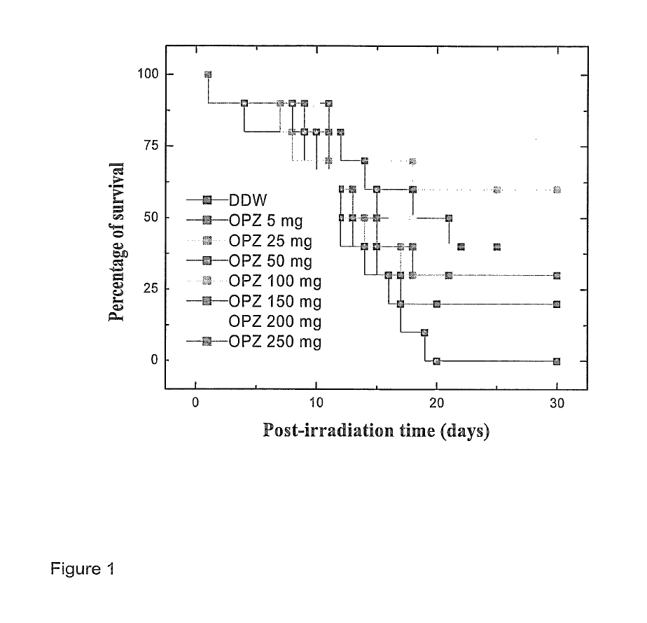

The pre-treatment of mice with various doses of 5-[2-pyrazinyl]-4-methyl-1,2-3-thione either delayed or reduced the severity of symptoms of radiation sickness. The onset of radiation-induced mortality was also delayed in the 5-[2-pyrazinyl]-4-methyl-1,2-3-thione and irradiation groups when compared with the CMC and irradiation group. The longest delay was observed for the 100 mg/kg 5-[2-pyrazinyl]-4-methyl-1,2-3-thione treated group where the first death was observed on day 11 post-irradiation (FIG. 1), indicating complete protection from gastrointestinal syndrome, whereas the shortest delay was observed for the 5 mg/kg 5-[2-pyrazinyl]-4-methyl-1,2-3-thione treatments where the first death occurred on day 7 post-irradiation (FIG. 1). This delay in mortality was also observed for other doses of 5-[2-pyrazinyl]-4-methyl-1,2-3-thione.

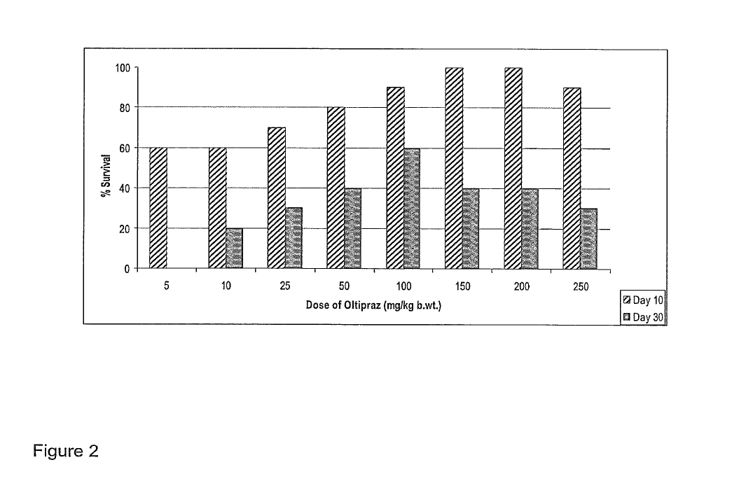

Treatment of mice with different doses of 5-[2-pyrazinyl]-4-methyl-1,2-3-thione protected against the radiation-induced gastrointestinal tract death, as evidenced by an increase in the ten-day survival of mice for all doses of the 5-[2-pyrazinyl]-4-methyl-1,2-3-thione treated group (FIG. 2). Administration of 150 mg/kg and 200 mg/kg 5-[2-pyrazinyl]-4-methyl-1,2-3-thione did not cause any mortality within 10 days of irradiation (FIG. 2). Analysis of thirty-day survival revealed an 5-[2-pyrazinyl]-4-methyl-1,2-3-thione dose-dependent increase in survival of irradiation animals with doses increasing up to 100 mg/kg, where the highest survival of 60% was observed as compared to the CMC and irradiation group where no survivors were reported (FIG. 2). An increase in drug dose to 150 and 200 mg resulted in 20% reduction in animal survival, whereas this reduction in survival was 30% for 250 mg/kg when compared with the 100 mg/kg 5-[2-pyrazinyl]-4-methyl-1,2-3-thione and irradiation group (FIG. 2). The lowest doses of 10, 25 and 50 mg/kg 5-[2-pyrazinyl]-4-methyl-1,2-3-thione also increased the survival by 20, 30 and 40% respectively when compared with the CMC and irradiation group where no survivors were observed. A significant elevation in survival was observed only in animals that received 50, 100, 150 and 200 mg/kg 5-[2-pyrazinyl]-4-methyl-1,2-3-thione before exposure to 10 Gy (p>0.05).

This example demonstrates that 5-[2-pyrazinyl]-4-methyl-1,2-3-thione, administered orally, protected mice against radiation-induced sickness and mortality. The optimum protective dose was found to be 100 mg/kg when compared to other doses as it increased survival by 60% when compared to a non-5-[2-pyrazinyl]-4-methyl-1,2-3-thione treated irradiated control.

### Example 2

### Evaluation of Radioprotective Efficacy of N⁶-Isopentenyl Adenosine

This experiment evaluated the efficacy and safety of N⁶-Isopentenyl Adenosine (also known as 6-gamma-Dimethyl Allyl Amino Purine Ribose (DAPR)) as a radioprotective agent.

### Acute Toxicity Studies

The animals were allowed to fast for 18 hours and were then administered with 0, 100, 200, 400, 500, 600, 700, 800, 1000, 1250, 1500, 1750 or 2000 mg/kg of DAPR and observed for 14 days post-drug treatment.

### Treatment Group 1—CMC and Irradiation

Animals of this group received 0.5% carboxymethyl cellulose (CMC) orally before exposure to 10 Gy of gamma irradiation.

### Treatment Group 2—DAPR and Irradiation

Animals of this group were treated with 1, 5, 10, 25, 50, 100, 150, 200 or 250 mg/kg body weight of DAPR orally once before exposure to 10 Gy of gamma radiation.

### Irradiation Procedure

One hour after the administration of CMC or DAPR, the immobilized animals, achieved by inserting cotton plugs in the restrainer, were whole-body exposed to ⁶⁰Co gamma radiation (Theratron, Atomic Energy Agency, Canada) in a specially designed well-ventilated acrylic box. A batch of ten animals was irradiated each time at a dose rate of 1.33 Gy/min.

### Results

### Acute Toxicity Studies

The animals receiving different doses of DAPR did not show any signs of toxicity up 2 g/kg and not a single mortality was observed up to 14 days. Therefore DAPR up to 2 g was considered completely safe for administration. Higher doses could not be evaluated owing to problems in drug dissolution.

Initially 1, 5 and 10 mg/kg DAPR were also evaluated. However, there was no alteration in the survival after irradiation. Therefore, these doses were abandoned in the subsequent evaluation. The radioprotective efficacy of DAPR was evaluated by treating mice with 0, 25, 50, 100, 150, 200 and 250 mg/kg body weight DAPR before whole-body exposure to 10 Gy gamma radiation.

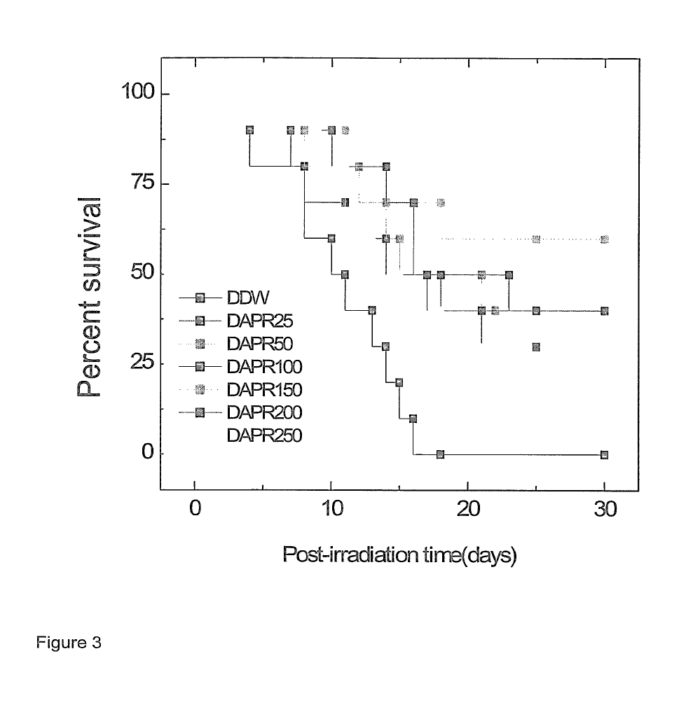

After irradiation, the animals were monitored daily for 30 days for the development of symptoms of radiation sickness and mortality. Exposure of the CMC and irradiation group to 10 Gy induced symptoms of severe radiation sickness, such as a reduction in food and water intake, irritability, lethargy, body weight loss, diarrhoea, lacrimation, facial edema, emaciation and epilation. The first mortality in the CMC and irradiation group was observed at day 4 and all of the irradiated animals died by day 18 post-irradiation (FIG. 3).

The pre-treatment of mice with various doses of DAPR either delayed or reduced the severity of symptoms of radiation sickness. The onset of radiation-induced mortality was also delayed in the DAPR and irradiation groups when compared with the CMC and irradiation group. The longest delay was observed for 150 mg/kg DAPR treated group where the first death was observed on day 11 post-irradiation (FIG. 3), indicating complete protection from gastrointestinal syndrome, whereas the shortest delay was observed for the 25 mg/kg DAPR treatments, where the first death occurred on day 7 post-irradiation (FIG. 3). This delay in mortality was also observed for other doses of DAPR.

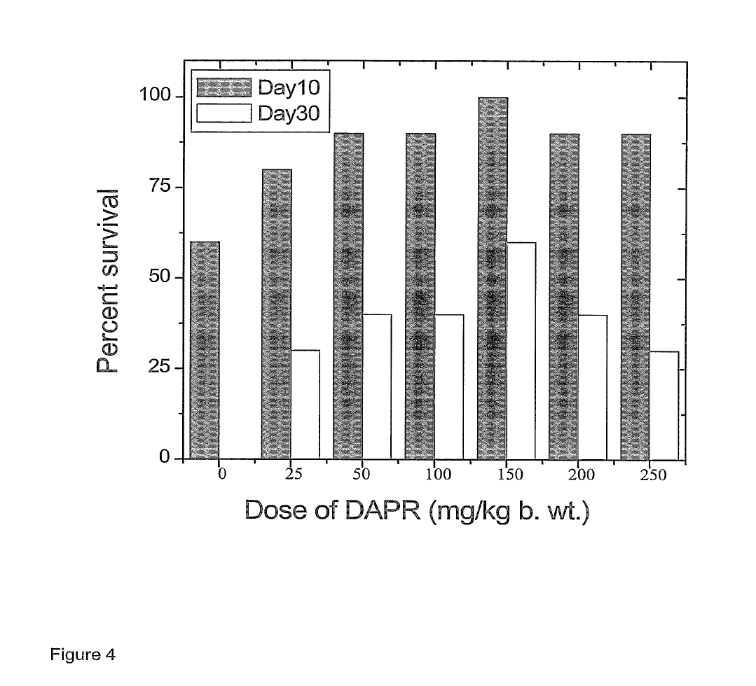

Treatment of mice with various doses of DAPR protected animals against the radiation-induced gastrointestinal tract death, as evidenced by an increase in the ten-day survival of mice for all doses of the DAPR treated group (FIG. 4). Administration of 150 mg/kg DAPR did not cause any mortality within 10 days of irradiation (FIG. 4). Analysis of thirty-day survival revealed a DAPR dose-dependent increase in survival of irradiation animals with doses increasing up to 150 mg/kg, where the highest survival of 60% was observed as compared to the CMC and irradiation group, where no survivors were reported (FIG. 4). An increase in drug dose to 200 and 250 mg resulted in 20% and 30% reductions respectively in survival when compared with the 150 mg/kg DAPR and irradiation group (FIG. 4). The lowest doses of 25 mg/kg DAPR also increased the survival by 30% respectively when compared with the CMC and irradiation group where no survivor's were observed. A significant elevation in survival was observed only in animals that received 50, 100, 150 and 200 mg/kg DAPR before exposure to 10 Gy (p>0.05).

This example demonstrates that DAPR is completely safe up to 2 g/kg as no toxic side effects could be observed and all doses of DAPR administered orally protected mice against the radiation-induced sickness and mortality. However, the optimum protective dose was found to be 150 mg/kg when compared to other doses as it increased the survival by 60%.

### Example 3

### Radioprotective effects of 5-[2-pyrazinyl]-4-methyl-1,2-3-thione (oltipraz)

This experiment evaluated the efficacy and safety of 5-[2-pyrazinyl]-4-methyl-1,2-3-thione (oltipraz) as a radioprotective agent.

### Animals

Male Swiss albino mice (Mus musculus), 6-8 weeks old with 25±3 g body weight from an inbred colony (obtained from Hamadard University, Delhi, India) were used for the present study. Animals were maintained under controlled conditions of temperature and light in an animal house and were provided with standard mice feed (procured from Hindustan Lever's Ltd. Delhi, India) and water ad libitum.

### Irradiation

Cobalt teletherapy unit (ATC-C9) at the Cancer treatment centre, Radiotherapy Department, SMS Medical College & Hospital, Jaipur, India, was used for irradiation. Unanaesthetised animals were restrained in well-ventilated perspex boxes and exposed whole-body to gamma radiation at the distance (SSD) of 77.5 cm from the source to deliver the dose-rate of 1.33 Gy/min.

### Acute Drug Toxicity

To determine the acute toxicity of oltipraz (5-[2-pyrazinyl]-4-methyl-1,2-3-thione), the animals were divided into 4 groups of 10 each and oltipraz (5-[2-pyrazinyl]-4-methyl-1,2-3-thione) was given orally to them at the concentration of 50, 100, 200 or 400 kg/body weight/day for 2 consecutive days. The mice were observed continuously for 30 days to determine the toxicity of oltipraz (5-[2-pyrazinyl]-4-methyl-1,2-3-thione) in the form of mortality or any other sign if present.

### Determination of Optimum Dose of Oltipraz (5-[2-Pyrazinyl]-4-Methyl-1,2-3-Thione) Against Radiation

For the selection of an optimum dose of oltipraz (5-[2-pyrazinyl]-4-methyl-1,2-3-thione) against radiation, animals were given 50, 100, 200 or 400 mg/kg body weight/day oltipraz (5-[2-pyrazinyl]-4-methyl-1,2-3-thione) for 2 consecutive days. Thirty minutes after the last administration, the animals were exposed to 8 Gy gamma radiation. Survival of the animals was recorded for 30 days after irradiation. The reduced glutathione (GSH) and lipid peroxidation (LPO) levels in liver and blood were estimated after 30 minutes of radiation exposure.

### Reduced Glutathione (GSH) Assay

The hepatic level of reduced glutathione (GSH) was determined as per the standard method. GSH content in blood was measured Spectrophotometrically using Ellman's reagent (DTNB) as a colouring reagent. The absorbance was read at 412 nm using a UV-VIS Systronics Spectrophotometer.

### Lipid Peroxidation (LPO) Assay

The lipid peroxidation level in liver and serum was measured in terms of Thiobarbituric Acid Reactive Substances [TBARS]. The absorbance was read at 532 nm.

### Dose Reduction Factor (DRF)

The protective capacity of an agent (chemical or plant extract) is expressed as dose reduction factor (DRF). It can be calculated by dividing the LD_{50/30} of experimental animals by LD_{50/30} of control animals.

### Control Group (Irradiation Alone)

These animals were exposed to 6, 8 and 10 Gy of Gamma rays and observed for 30 days to record mortality and signs of radiation sickness.

### Experimental Group (oltipraz (5-[2-pyrazinyl]-4-methyl-1,2-3-thione) and Irradiation)

Animals of this group were given oltipraz (5-[2-pyrazinyl]-4-methyl-1,2-3-thione) orally at the dose level of 100 mg/kg body weight/day for 2 consecutive days and exposed to 6, 8, and 10 Gy of gamma rays after the last administration. The animals were observed for 30 days and radiation sickness and mortality were recorded in a similar manner as for the control group.

### Body Weight

The general condition and body weights of the mice in all groups were observed daily. The percent change in body weight in each group of mice was recorded every day by dividing the average body weight of those mice on the first day of treatment.

### Endogenous Spleen Colony Assay

The endogenous spleen colony assay was done according to the method of Till and McCulloch. Endogenous spleen colony forming units (CFU-S) were determined on day 10 after irradiation. Animals were sacrificed by cervical dislocation. Their spleens were removed, weighed and fixed in Bouin's fixative. Grossly visible nodules on the surface of the spleen were counted with the naked eye.

### Survival Assay

Mice of both groups (control as well as experimental) exposed to 6, 8 and 10 Gy gamma radiation were checked daily for 30 days and the percentage of mice surviving 30 days of exposure against each radiation dose was used to construct survival-dose response curves.

### Quantitative Changes in Spleen

The weight of the spleen at each autopsy interval (day 1, 3, 7, 10, 14 and 30 post-irradiation) was determined to study changes.

### Statistical Analysis

The results obtained are expressed as mean±SE. Student's “t” test was used to make a statistical comparison between the groups. Significance levels were set at P<0.05, P<0.01 and P<0.001. Regression analysis was done to obtain LD_{50/30} values and to determine the dose reduction factor (DRF).

### Chromosomal Aberration Analysis

Cytogenetic damage in the bone marrow cells was studied by chromosomal aberration analysis at the end of the experiments. All animals were injected intra-peritoneally (i.p.) with 0.025% colchicine and sacrificed 2 hours later by cervical dislocation. Both femurs were dissected out. Metaphase plates were prepared by the air drying method. Bone marrow from the femur was aspirated, washed in saline, treated hypotonically (0.6% sodium citrate), fixed in 3:1 methanol:acetic acid, dried and stained with 4% Giemsa (Sigma, USA). Chromosomal aberrations were scored under a light microscope. A total of 400 metaphase plates were scored per animal. Different types of aberration-like chromatid breaks, chromosome breaks, fragments, rings, exchanges and dicentrics were scored. When breaks involved both chromatids, it was termed a “chromosome type” aberration, while “chromatid type” aberrations involved only one chromatid. If the deleted portion had no apparent relation to a specific chromosome, it was called a fragment.

### Results

The radioprotective efficacy of oltipraz (5-[2-pyrazinyl]-4-methyl-1,2-3-thione) against radiation-induced sickness, changes in body weight, spleen colonies and animal survival was studied in Swiss albino mice. Treatment with oltipraz (5-[2-pyrazinyl]-4-methyl-1,2-3-thione) for two consecutive days in mice did not produce any toxic effect. Rather, these animals showed an increase in body weight at 30 days as compared to sham irradiated (normal) animals.

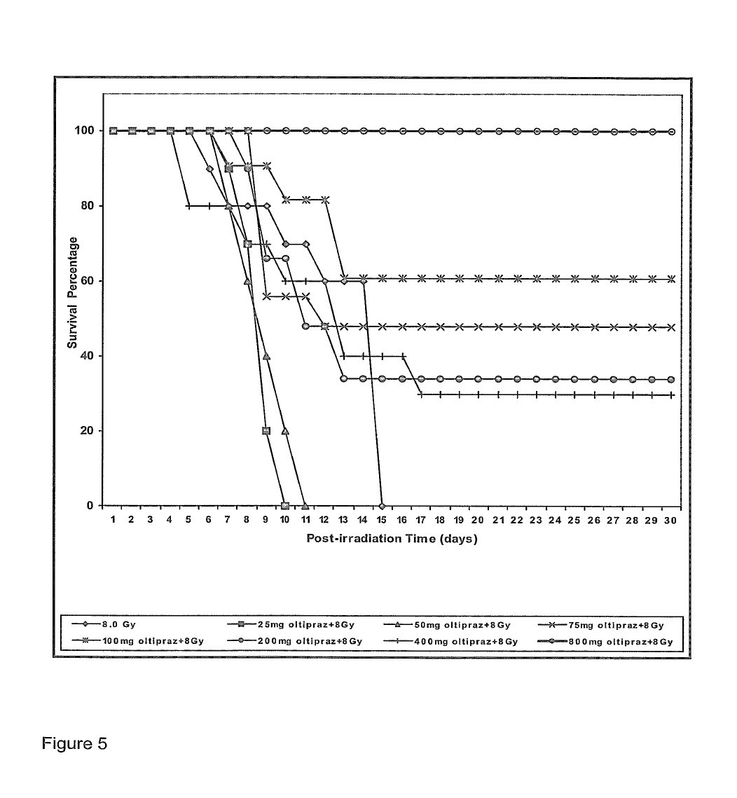

The optimum dose of oltipraz (5-[2-pyrazinyl]-4-methyl-1,2-3-thione)exhibiting maximum radioprotection was found to be 100 mg/kg body weight/day for 2 consecutive days before irradiation (FIG. 5).

No significant variation in the GSH contents of the liver and blood was observed in normal and oltipraz (5-[2-pyrazinyl]-4-methyl-1,2-3-thione) treated animals (Table 2). However, a significant decrease in GSH content was observed in control animals (irradiation alone), whereas experimental animals showed a significant increase in GSH content (blood as well as liver) at various concentrations of oltipraz (5-[2-pyrazinyl]-4-methyl-1,2-3-thione) as compared to the control (Table 2). The maximum increase in GSH content was observed in the animals pre-treated with 100 mg/kg body weight/day oltipraz (5-[2-pyrazinyl]-4-methyl-1,2-3-thione).

An increase in TBARS level in the liver and serum was also evident in control animals as compared to normal animals, although no significant difference was noticed in such levels in normal and oltipraz (5-[2-pyrazinyl]-4-methyl-1,2-3-thione) treated animals (Table 2). A significant dose-dependent decrease was registered in oltipraz (5-[2-pyrazinyl]-4-methyl-1,2-3-thione) pre-treated irradiated animals. However, the maximum decline in LPO level was measured in the animals pre-treated with 100 mg/kg body weight/day oltipraz (5-[2-pyrazinyl]-4-methyl-1,2-3-thione).

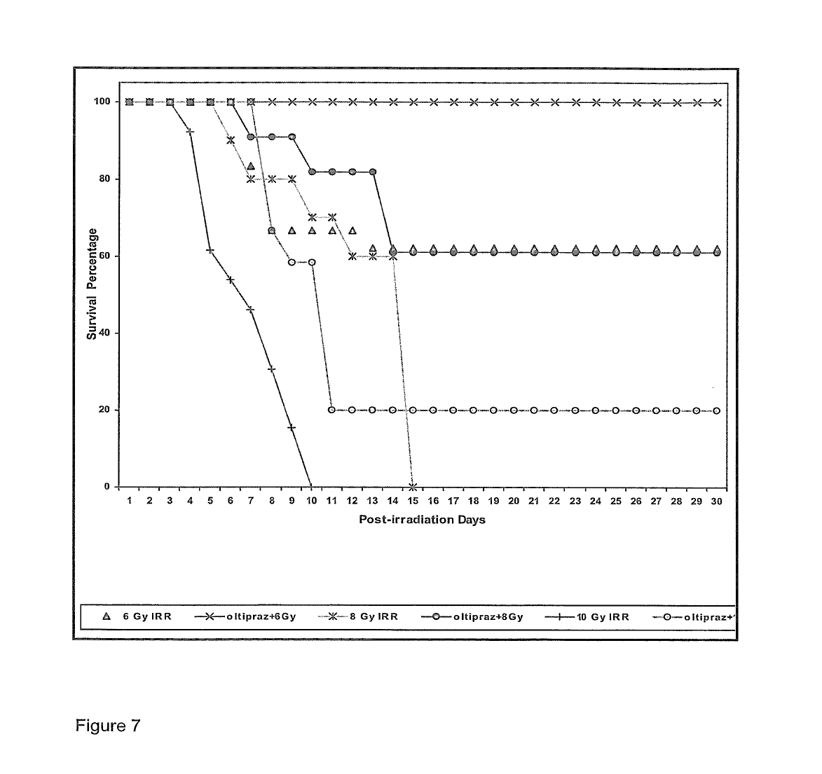

In the present study, it was observed that pre-treatment with oltipraz (5-[2-pyrazinyl]-4-methyl-1,2-3-thione) enhanced the survival of mice exposed to different doses of gamma radiation (FIG. 5). Signs of radiation sickness such as lethargy, diarrhoea, loss of body weight, ruffled hairs, epilation, facial edema, and loss of appetite were observed in the animals exposed to different doses of gamma-radiation (control). The severity of the radiation sickness was dose-dependent and 38% of the animals died within 30 days post irradiation with 6 Gy, whereas 100% mortality was observed on day 14 and day 10 in animals of control groups after exposure to 8 and 10 Gy respectively (FIG. 7). No radiation sickness was observed in the animals treated with 100 mg/kg body weight/day oltipraz (5-[2-pyrazinyl]-4-methyl-1,2-3-thione) before exposure to 6 Gy. However, the severity of radiation sickness was much less in comparison to their respective controls after irradiation with 8 and 10 Gy. The survivability in 6 Gy experimental groups was 100% but it decreased to 61% and 20% in experimental groups after irradiation with 8 and 10 Gy respectively (FIG. 7).

Regression analysis of survival data showed 6.24 and 8.82 Gy LD_{50/30} values for control and experimental animals respectively. On the basis of LD_{50/30} values, a DRF was calculated as 1.25.

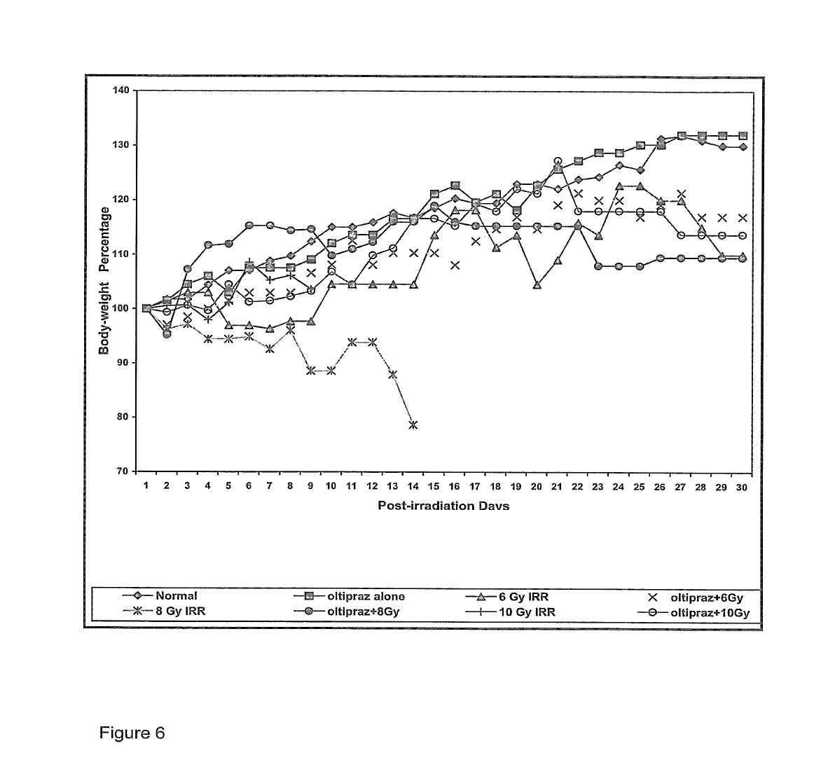

Maximum body weight loss was 24% and minimum loss was 13.5% in control groups whereas in experimental groups it was 22.05 and 1.7% in their respective groups. Not only this, but the experimental animals showed 17% (6 Gy), 9.5% (8 Gy) and 13.7% (10 Gy) increase in their body weight from their initial body weights at day-30 post-irradiation (FIG. 6).

The protective effect of oltipraz (5-[2-pyrazinyl]-4-methyl-1,2-3-thione) against radiation injury to hematopoietic tissue was assessed by the endogenous spleen colony assay and spleen weight changes. It was observed that oltipraz (5-[2-pyrazinyl]-4-methyl-1,2-3-thione) pre-treatment of mice increased the number of spleen colonies significantly over that of the irradiation alone group (Table 3). The pattern of spleen weight change was similar in all the control groups (irradiation alone) up to day 7 after irradiation, but the decrease in spleen weight was found to be dose-dependent, that is the higher the radiation dose, the greater the weight loss. The maximum weight loss was observed at day 7, after the increase in the tissue weight was registered. Further, an increase in the weight of the spleen was observed which was greater than normal on day 14 and attained normal value at day 30 in animals irradiated at 6 Gy. No animal could survive beyond day 14 (8 Gy) and day 10 (10 Gy) for the exposed groups (FIG. 7). The spleen weight in oltipraz (5-[2-pyrazinyl]-4-methyl-1,2-3-thione) treated and irradiated (experimental) animals decreased until day 7 but the decrease was significantly less as compared to the control group at each autopsy interval. After day 7, a gradual increase was observed which attained almost normal value by day 30.

### Chromosomal Study

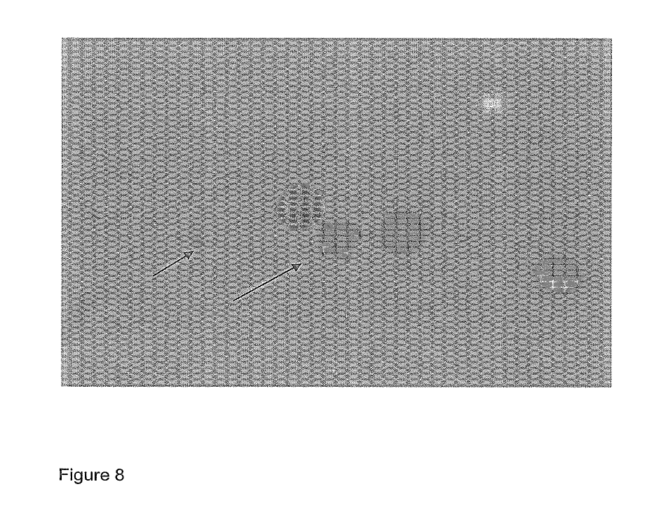

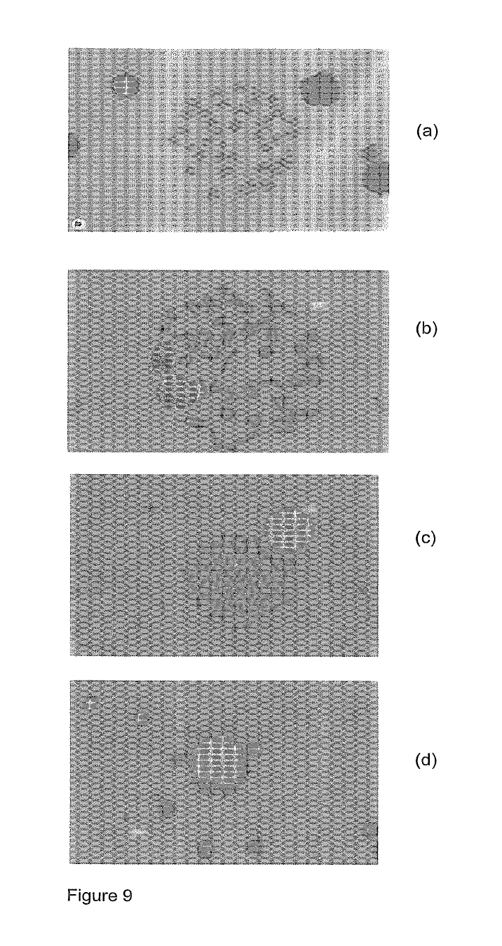

Oral administration of 5-[2-pyrazinyl]-4-methyl-1,2-3-thione (100 mg/kg body weight/day) before exposure to gamma radiation was found to be effective in protecting against chromosomal damage in bone marrow of Swiss albino mice (FIG. 9, Table 5 and Table 6). Animals exposed to 8 Gy gamma radiation showed chromosomal aberrations in the form of chromatid breaks, chromosome breaks, centric rings, dicentrics, exchanges and acentric fragments. There was a significant increase in the frequency of aberrant cells at 6 hours after irradiation. Maximum aberrant cells were observed at 12 hours post irradiation autopsy time. Further, the frequency of aberrant cells showed decline at late post-irradiation autopsy time. However, in the animals pre-treated with 5-[2-pyrazinyl]-4-methyl-1,2-3-thione, there was a significant decrease in the frequency of aberrant cells as compared to the irradiated control. There was a significant increase in the number of micronuclei in 8 Gy irradiated mice. However, there was a significant decrease in the number of micronuclei in the animals pre-treated with 5-[2-pyrazinyl]-4-methyl-1,2-3-thione (Table 4 and FIG. 8).

TABLE 1
Radiomodulatory influence of 5-[2-pyrazinyl]-4-methyl-
1,2-3-thione on 30-day survival of Swiss albino mice

| | 30-Day Survival | | | |
|---|---|---|---|---|
| GROUP | Percentage | | LD_{50/30} | DRF |
| Control | 6 Gy | 62 | 6.35 Gy | 1.34 |
| [Radiation alone] | 8 Gy | 0 | (y = 144.66-15.5x) | |
| | 10 Gy | 0 | | |
| Experimental | 6 Gy | 100 | 8.51 Gy | |
| [5-[2-pyrazinyl]-4- | 8 Gy | 61 | (y = 220.33-20x) | |
| methyl-1,2-3-thione + | 10 Gy | 20 | | |
| Radiation] | | | | |

TABLE 2
Radiomodulatory influence of 5-[2-pyrazinyl]-4-methyl-1,2-3-
thione on GSH and LPO levels in liver and blood of Swiss albino mice

| | Liver | | Blood | |
|---|---|---|---|---|
| Treatment | GSH | LPO | GSH | LPO |
| Groups | (μmole/gm) | (nmol/mg) | (μg/ml) | (nmol/ml) |
| Normal | 64.62 ± 1.60 | 2.52 ± 0.17 | 4.02 ± 0.16 | 1.15 ± 0.11 |
| 5-[2-pyrazinyl]-4- | 65.68 ± 1.48 | 2.42 ± 0.14 | 4.18 ± 0.18 | 1.10 ± 0.10 |
| methyl-1,2-3-thione | | | | |
| alone | | | | |
| 6 Gy IRR | 46.26 ± 1.32^{c} | 4.84 ± 0.18^{c} | 2.84 ± 0.10^{c} | 2.85 ± 0.18^{c} |
| 5-[2-pyrazinyl]-4- | 52.44 ± 1.54^{b} | 3.22 ± 0.12^{c} | 3.02 ± 0.12^{c} | 2.44 ± 0.16^{c} |
| methyl-1,2-3-thione ± | | | | |
| 6 Gy | | | | |
| 8 Gy IRR | 36.28 ± 1.24^{c} | 6.82 ± 0.26^{c} | 2.21 ± 0.14^{c} | 4.10 ± 0.24^{c} |
| 5-[2-pyrazinyl]-4- | 54.62 ± 1.72^{c} | 3.80 ± 0.14^{c} | 2.88 ± 0.11^{b} | 3.28 ± 0.16^{a} |
| methyl-1,2-3-thione ± | | | | |
| 8 Gy | | | | |
| 10 Gy IRR | 29.82 ± 1.18^{c} | 8.52 ± 0.24^{c} | 2.11 ± 0.10^{c} | 4.96 ± 0.22^{c} |
| 5-[2-pyrazinyl]-4- | 44.38 ± 1.42^{c} | 5.22 ± 0.27^{c} | 2.40 ± 0.12 | 3.68 ± 0.18^{b} |
| methyl-1,2-3-thione ± | | | | |
| 10 Gy | | | | |

Significance levels:
^{a}p < 0.05,
^{b}p < 0.005 and
^{c}p < 0.001.

TABLE 3
Spleen response on day 10 post-irradiation in absence and
presence of 5-[2-pyrazinyl]-4-methyl-1,2-3-thione treatment
in Swiss albino mice

Spleen Response

| Treatment | Spleen Weight | Number of macroscopic |
|---|---|---|
| Groups | (mg) | colonies |
| Normal | 44.20 ± 1.22 | 0.00 ± 0.00 |
| 6 Gy IRR | 34.68 ± 1.32^{c} | 4.84 ± 0.18^{c} |
| 5-[2-pyrazinyl]-4- | 42.44 ± 1.24^{b} | 10.22 ± 0.62 |
| methyl-1,2-3- | | |
| thione ± 6 Gy | | |
| 8 Gy IRR | 26.28 ± 0.84^{c} | 6.82 ± 0.26^{c} |
| 5-[2-pyrazinyl]-4- | 32.62 ± 1.02^{b} | 13.80 ± 0.84^{c} |
| methyl-1,2-3- | | |
| thione ± 8 Gy | | |
| 10 Gy IRR | ns | ns |
| 5-[2-pyrazinyl]-4- | 34.38 ± 1.20^{c} | 15.22 ± 0.77^{c} |
| methyl-1,2-3- | | |
| thione ± 10 Gy | | |

Significance levels:
^{a}p < 0.05,
^{b}p < 0.005 and
^{c}p < 0.001.
NS = Not survived.

TABLE 4
Micronucleus frequency in bone marrow cells of Swiss albino
mice with or without 5-[2-pyrazinyl]-4-methyl-1,2-3-thione
treatment following 8 Gy gamma radiation

| | Number of Mn/1000 |
|---|---|
| Group | Cells |
| Control | 22.16 ± 1.24^{c} |
| Experimental | 6.58 ± 0.64^{c} |
| Normal | 0.32 ± 0.04 |
| 5-[2-pyrazinyl]-4- | 0.28 ± 0.01 |
| methyl-1,2-3-thione | |
| alone | |

Each value represents Mean +/− SE
Control = 8.0 Gy gamma rays
Experimental = 5-[2-pyrazinyl]-4-methyl-1,2-3-thione + 8.0 Gy gamma rays
Normal = no treatment
Significance levels:
^{a}p < 0.05,
^{b}p < 0.005 and
^{c}p < 0.001.

TABLE 5
Frequencies of chromosomal aberrations in Swiss albino mice
with or without 5-[2-pyrazinyl]-4-methyl-1,2-3-thione treatment following 8
Gy gamma radiation

| | Chromatid | Chromosome | Centric | Dicentrics | Exchanges | Fragments |
|---|---|---|---|---|---|---|
| Group | breaks (%) | breaks (%) | rings (%) | (%) | (%) | (%) |
| Control | 5.88 ± 1.12^{c} | 2.29 ± 0.32^{c} | 1.78 ± 0.32^{c} | 1.88 ± 0.38^{b} | 2.60 ± 0.46^{c} | 98.6 ± 4.66^{c} |
| Experimental | 3.18 ± 0.44^{a} | 1.04 ± 0.26^{b} | 1.26 ± 0.28 | 1.10 ± 0.14 | 1.18 ± 0.32^{b} | 28.8 ± 3.20^{c} |
| Normal | 0.16 ± 0.01 | 0.00 ± 0.00 | 0.00 ± 0.00 | 0.00 ± 0.00 | 0.00 ± 0.00 | 1.10 ± 0.05 |
| 5-[2- | 0.14 ± 0.01 | 0.00 ± 0.00 | 0.00 ± 0.00 | 0.00 ± 0.00 | 0.00 ± 0.00 | 0.85 ± 0.04^{b} |
| pyrazinyl]- | | | | | | |
| 4- | | | | | | |
| methyl- | | | | | | |
| 1,2-3- | | | | | | |
| thione | | | | | | |
| alone | | | | | | |

Each value represent Mean +/− SE. Total 400 metaphases were scored per animal.
Significance levels:
^{a}p < 0.05,
^{b}p < 0.005 and
^{c}p < 0.001.

TABLE 6
Frequencies of chromosomal aberrations in Swiss albino mice
with or without 5-[2-pyrazinyl]-4-methyl-1,2-3-thione treatment following 8
Gy gamma radiation

| | | | | | Aberrations |
|---|---|---|---|---|---|
| | | | | Total | per |
| | Pulverized | Polyploidy | Aberrant | aberrations | damaged |
| Group | cells (%) | (%) | cells (%) | (%) | cell |
| Control | 5.24 ± 0.08^{c} | 3.28 ± 0.06^{c} | 56.14 ± 2.16^{c} | 164.82 ± 8.28^{c} | 2.92 ± 0.22^{c} |
| Experimental | 1.28 ± 0.02^{c} | 0.36 ± 0.01^{c} | 17.20 ± 1.20^{c} | 38.10 ± 3.66^{c} | 2.21 ± 0.20^{a} |
| Normal | 0.00 ± 0.00 | 0.12 ± 0.02 | 0.52 ± 0.03 | 0.82 ± 0.05 | 1.57 ± 0.04 |
| 5-[2- | 0.00 ± 0.00 | 0.18 ± 0.02 | 0.61 ± 0.04 | 0.74 ± 0.02 | 1.21 ± 0.03^{c} |
| pyrazinyl]-4- | | | | | |
| methyl-1,2-3- | | | | | |
| thione alone | | | | | |

Each value represent Mean +/− SE. Total 400 metaphases were scored per animal.
Significance levels:
^{a}p < 0.05,
^{b}p < 0.005 and
^{c}p < 0.001.

### Example 4

### Evaluation of 5-[2-pyrazinyl]-4-methyl-1,2-3-thione (oltipraz) in Combination with Radiation for Efficacy in the Prevention of Weight Loss and In the Reduction of Tumour Growth

The objective of this study was to evaluate the efficacy of 5-[2-pyrazinyl]-4-methyl-1,2-3-thione (oltipraz) in inhibiting tumour growth and preventing weight loss using a NCI H146 small cell lung cancer model in nude mice, both as a mono-therapy and in conjunction with radiotherapy.

### Study Design

Ninety-six (96) female nude mice (nu⁺/nu⁺) were randomly assigned into 8 treatment groups. Each mouse was inoculated with 1×10⁶ NCI-H146 (H146) small cell lung cancer cells in a volume of 0.05 mL on their lower left flank with Matrigel. Treatment began once tumors reached a volume of 75-125 mm³. The groups were treated with vehicle, radiation, 5-[2-pyrazinyl]-4-methyl-1,2-3-thione or radiation and 5-[2-pyrazinyl]-4-methyl-1,2-3-thione as detailed in Table 7.

TABLE 7
Study Design

| | | Tumour | RT | Drug | | |
|---|---|---|---|---|---|---|
| | No. of | cell | Days | Treatment | | Dose |
| Group | animals | inoculum | 2 and 4 | & Dosing | Route | Schedule |
| 1 | 12 | 1 × 10⁶ | none | Vehicle | po | qd, |
| | | | | | | days 1 |
| | | | | | | and 3 |
| 2 | 12 | 1 × 10⁶ | none | 5-[2- | po | qd, |
| | | | | pyrazinyl]- | | days 1 |
| | | | | 4-methyl- | | and 3 |
| | | | | 1,2-3- | | |
| | | | | thione | | |
| | | | | 50 mg/kg | | |
| 3 | 12 | 1 × 10⁶ | none | 5-[2- | po | qd, |
| | | | | pyrazinyl]- | | days 1 |
| | | | | 4-methyl- | | and 3 |
| | | | | 1,2-3- | | |
| | | | | thione | | |
| | | | | 100 mg/kg | | |
| 4 | 12 | 1 × 10⁶ | none | 5-[2- | po | qd, |
| | | | | pyrazinyl]- | | days 1-20 |
| | | | | 4-methyl- | | |
| | | | | 1,2-3- | | |
| | | | | thione | | |
| | | | | 50 mg/kg | | |
| 5 | 12 | 1 × 10⁶ | 2 Gy | Vehicle | po | qd, |
| | | | focal | | | days 1 |
| | | | | | | and 3 |
| 6 | 12 | 1 × 10⁶ | 2 Gy | 5-[2- | po | qd, |
| | | | focal | pyrazinyl]- | | days 1 |
| | | | | 4-methyl- | | and 3 |
| | | | | 1,2-3- | | |
| | | | | thione | | |
| | | | | 50 mg/kg | | |
| 7 | 12 | 1 × 10⁶ | 2 Gy | 5-[2- | po | qd, |
| | | | focal | pyrazinyl]- | | days 1 |
| | | | | 4-methyl- | | and 3 |
| | | | | 1,2-3- | | |
| | | | | thione | | |
| | | | | 100 mg/kg | | |
| 8 | 12 | 1 × 10⁶ | 2 Gy | 5-[2- | po | qd, |
| | | | focal | pyrazinyl]- | | days 1-20 |
| | | | | 4-methyl- | | |
| | | | | 1,2-3- | | |
| | | | | thione | | |
| | | | | 50 mg/kg | | |

Initiation of drug treatment was designated day 1. Mice in groups 1 and 5 received vehicle by oral gavage once daily on days 1 and 3. Mice in groups 2, 3, 6 and 7 received 5-[2-pyrazinyl]-4-methyl-1,2-3-thione (50 mg/kg or 100 mg/kg) in vehicle once a day by oral gavage on days 1 and 3. Mice in groups 4 and 8 received 5-[2-pyrazinyl]-4-methyl-1,2-3-thione (50 mg/kg) in vehicle once a day by oral gavage on days 1 through 20. Mice in groups 5 to 8 received radiation. The radiation was given as 2 doses of 2 Gy/dose on days 2 and 4. This was accomplished by anesthetizing the mice in these groups with ketamine (100 mg/kg) and xylazine (5 mg/kg) and placing them under a lead shield such that the region of the flank with tumor exposed to the radiation. Radiation was delivered using a Philips 160 kV source at a focal distance of approximately 40 cm, and a dose rate of approximately 1.0 Gy/min. Tumours were measured on alternating days throughout the duration of the study. All mice were sacrificed on day 21 and remaining tumours were excised, measured, weighed, photographed and fixed in formalin for later analysis.

### Weights and Survival

All animals were weighed every day and their survival recorded, in order to assess possible differences in animal weight among treatment groups as an indication of possible toxicity resulting from the treatments. Any animals exhibiting a loss of >20% of starting weight during the course of the study were euthanized.

### Tissue Culture

H146 human lung cancer cells were obtained from ATCC. These cells were grown in DMEM supplemented with 10% Fetal Calf Serum (FCS), penicillin and streptomycin, and 2 mM L-Glutamine. Cells were sub-cultured by removing the medium, rinsing twice with sterile calcium- and magnesium-free phosphate buffered saline (PBS) and adding 1 to 2 ml of 0.25% trypsin, 0.03% EDTA solution. The flask was allowed to sit at 37° C. until the cells detached. Cells were then sub-cultured at a ratio of 1:3.

### Location(s) of Study Performance

The study was performed at Biomodels AAALAC accredited facility in Watertown Mass. IACUC approval for this study was obtained from Biomodels IACUC.

### Animals

Female nude mice which are homozygous for the nu gene (nu⁺/nu⁺) (Charles River Labs, strain code 088; Crl-NUFoxn1^{nu}), aged 5 to 6 weeks, with a mean pre-treatment body weight of 23.8 grams were used. Animals were individually numbered using an ear punch and housed in groups of 5-6 animals per cage. Animals were acclimatized prior to study commencement. During this period of at least 2 days, the animals were observed daily in order to reject animals that presented in poor condition. The nude mouse colony at Charles River Labs was founded with mice obtained from NIH, derived from a spontaneously occurring mutation that results in a complete lack of thymic epithelium and a significant reduction in fur and whiskers. The lack of thymic epithelium prevents the maturation of T-cells, resulting in a significant deficiency in the cell mediated immune response. These animals are generally regarded as being immunodeficient, and are susceptible to tumours that are not syngeneic.

### Housing

The study was performed in animal rooms provided with filtered air at a temperature of 70° F.+/−5° F. and 50%+/−20% relative humidity. Animal rooms were set to maintain a minimum of 12 to 15 air changes per hour. The room was on an automatic timer for a light/dark cycle of 12 hours on and 12 hours off with no twilight.

Sterilized Bed-O-Cobs® bedding was used. Bedding was changed a minimum of once per week.

Cages, tops, bottles, etc. were washed with a commercial detergent and allowed to air dry. Prior to use, these items were wrapped and autoclaved. A commercial disinfectant was used to disinfect surfaces and materials introduced into the hood. Floors were swept daily and mopped a minimum of twice weekly with a commercial detergent. Walls and cage racks were sponged a minimum of once per month with a dilute bleach solution. A cage card or label with the appropriate information necessary to identify the study, dose, animal number and treatment group marked all cages. The temperature and relative humidity were recorded during the study, and the records retained.

### Diet

Animals were fed with sterile Labdiet® 5053 (pre-sterilized) rodent chow and sterile water was provided ad libitum.

### Animal Randomization and Allocations

Mice were randomly and prospectively divided into eight (8) treatment groups prior to the initiation of treatment. Each animal was identified by ear punching corresponding to an individual number. A cage card was used to identify each cage and marked with the study number (CAN-01), treatment group number and animal numbers.

### Assessment of Results

Statistical differences between treatment groups were determined using Student's t-test, Mann-Whitney U test and chi-square analysis with a critical value of 0.05.

### Experimental Procedures

Tumours were measured once every two days with micro-calipers, and tumour volume was calculated as (length×width)³π/3. The tumour growth index (TGI) was calculated using the formula 100−(Vc*100/Vt), where Vc is the mean volume of the tumours in the control group and Vt is the mean volume of the tumours in the test group.

### Results

A total of four deaths were noted in this study. Three deaths were related to the anaesthesia used to immobilize the animals for radiation (2 on day 2 in groups 6 and 7, one on day 4 in group 7). The fourth death occurred on day 15 in the group treated with radiation plus 5-[2-pyrazinyl]-4-methyl-1,2-3-thione at 100 mg/kg on days 1 and 3 (group 7).

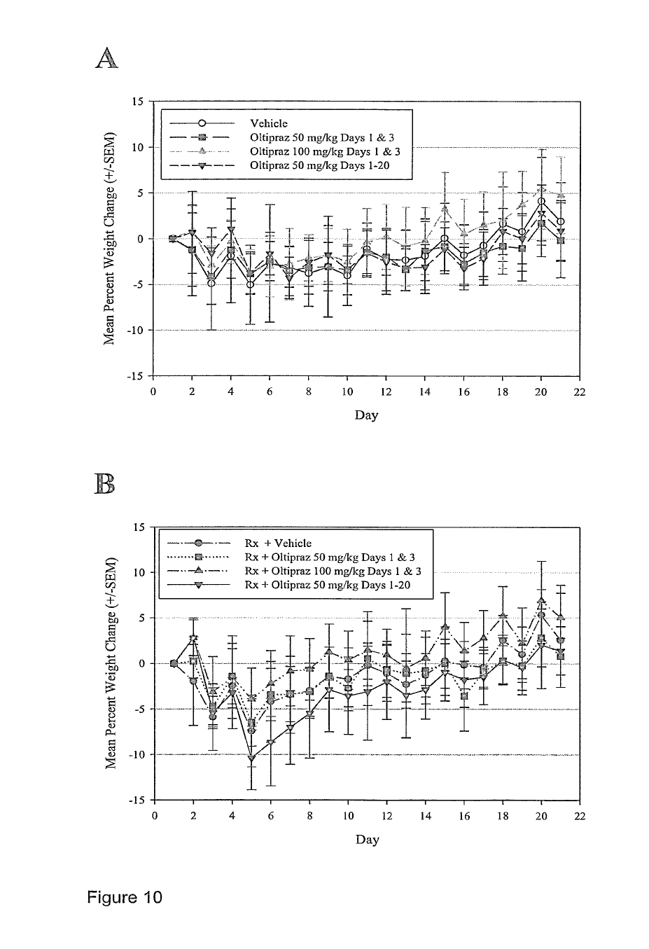

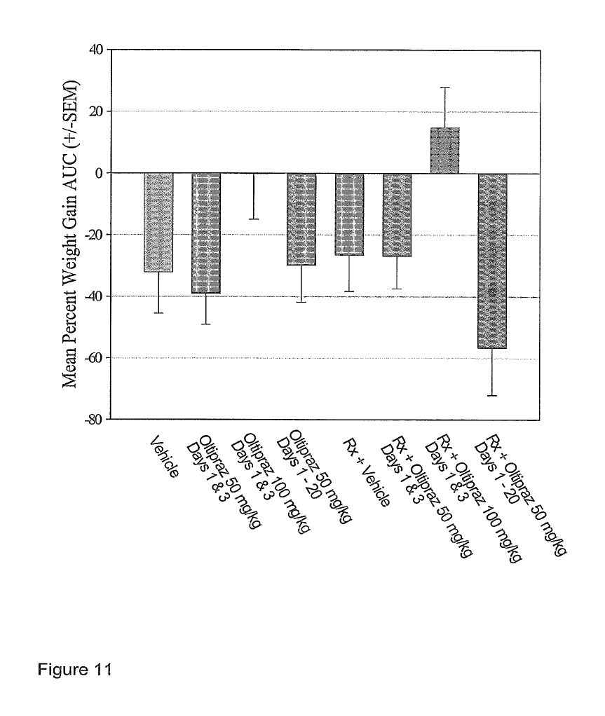

### Weight Loss (FIGS. 10 and 11)

The mean daily percentage weight change for each treatment group is shown in FIG. 10. The mice in the vehicle control group gained an average of 1.8% of their starting weight by Day 21. The mice treated with 5-[2-pyrazinyl]-4-methyl-1,2-3-thione 50 mg/kg on days 1 and 3 lost an average of 0.1% of their starting weight by Day 21. The mice treated with 5-[2-pyrazinyl]-4-methyl-1,2-3-thione 100 mg/kg on days 1 and 3 gained an average of 4.8% of their starting weight by Day 21. The mice treated with 5-[2-pyrazinyl]-4-methyl-1,2-3-thione 50 mg/kg on days 1 to 20 gained an average of 0.8% of their starting weight by Day 21. The mice receiving radiation plus vehicle gained 2.6% of their starting weight by Day 21. The mice treated with radiation plus 5-[2-pyrazinyl]-4-methyl-1,2-3-thione 50 mg/kg on days 1 and 3 gained an average of 0.8% of their starting weight by Day 21. The mice treated with radiation plus 5-[2-pyrazinyl]-4-methyl-1,2-3-thione 100 mg/kg on days 1 and 3 gained an average of 5.1% of their starting weight by Day 21. The mice treated with radiation plus 5-[2-pyrazinyl]-4-methyl-1,2-3-thione 50 mg/kg on days 1 to 20 gained an average of 1.4% of their starting weight by Day 21.

The significance of these differences was evaluated by calculating the mean area under the curve (AUC) for the percentage weight change for each animal and comparing the groups using a One-Way ANOVA test. There were no significant differences between the 5-[2-pyrazinyl]-4-methyl-1,2-3-thione treated groups and the vehicle control groups (P=0.153). There was a significant difference between the group treated with radiation plus 5-[2-pyrazinyl]-4-methyl-1,2-3-thione 100 mg/kg on days 1 and 3 and the group treated with radiation plus 5-[2-pyrazinyl]-4-methyl-1,2-3-thione 100 mg/kg on days 1 to 20 (P=0.003). The AUG data is shown in FIG. 11.

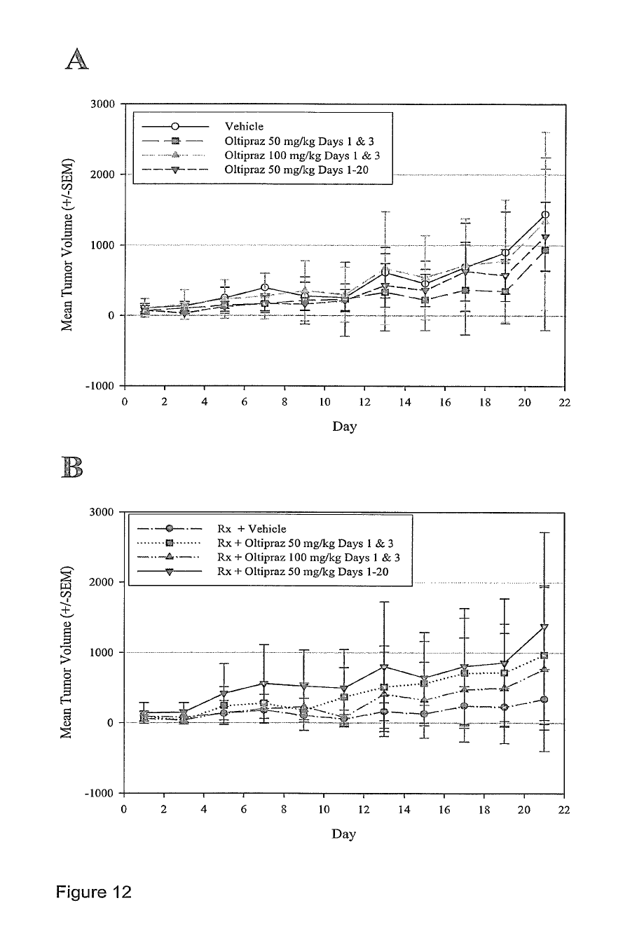

### Tumour Volumes (FIG. 12)

Tumour volumes were calculated from the length and width measurements taken on alternating days by calculating the mean radius (r), which was the sum of length and width divided by 4, and using the formula 4/3 πr³ to calculate the volume. The mean tumour volume data is shown in FIG. 12. The mean tumour volume for the vehicle control group increased from 109 mm³ on Day 1 to 1374 mm³ on Day 21. The mean tumour volume for the group treated with 5-[2-pyrazinyl]-4-methyl-1,2-3-thione at 50 mg/kg on days 1 and 3 increased from 72 mm³ on Day 1 to 940 mm³ on Day 21. The mean tumour volume for the group treated with 5-[2-pyrazinyl]-4-methyl-1,2-3-thione at 100 mg/kg on days 1 and 3 increased from 110 mm³ on Day 1 to 1341 mm³ on Day 21. The mean tumour volume for the group treated with 5-[2-pyrazinyl]-4-methyl-1,2-3-thione at 50 mg/kg on days 1 to 20 increased from 76 mm³ on Day 1 to 1130 mm³ on Day 21. The mean tumour volume for the radiation therapy plus vehicle control group increased from 92 mm³ on Day 1 to 339 mm³ on Day 21. The mean tumour volume for the group treated with radiation plus 5-[2-pyrazinyl]-4-methyl-1,2-3-thione at 50 mg/kg on days 1 and 3 increased from 93 mm³ on Day 1 to 971 mm³ on Day 21. The mean tumour volume for the group treated with radiation plus 5-[2-pyrazinyl]-4-methyl-1,2-3-thione at 100 mg/kg on days 1 and 3 increased from 63 mm³ on Day 1 to 769 mm³ on Day 21. The mean tumour volume for the group treated with radiation plus 5-[2-pyrazinyl]-4-methyl-1,2-3-thione at 50 mg/kg on days 1 to 20 increased from 140 mm³ on Day 1 to 1380 mm³ on Day 21.

Further analysis of the data was performed by calculating the mean area under the curve (AUC) for the tumour volume for each animal and comparing the groups using a One-Way ANOVA on ranks test. The overall analysis did not reveal significant differences between the 5-[2-pyrazinyl]-4-methyl-1,2-3-thione treated groups and the vehicle control groups (P=0.052). However individual group to group comparisons using the Mann-Whitney Rank sum test indicated that there was a significant difference between the group treated with radiation plus vehicle and the group treated with vehicle alone (P=0.004). In addition, there was a significant difference between the group treated with vehicle alone and the group treated with 5-[2-pyrazinyl]-4-methyl-1,2-3-thione at 50 mg/kg on days 1 and 3 (P=0.030). While it is clear that 5-[2-pyrazinyl]-4-methyl-1,2-3-thione does not have an additive effect when given in conjunction with radiation therapy, at least at the doses and dose schedules used in this study, it appears that 5-[2-pyrazinyl]-4-methyl-1,2-3-thione may be effective as a single agent.

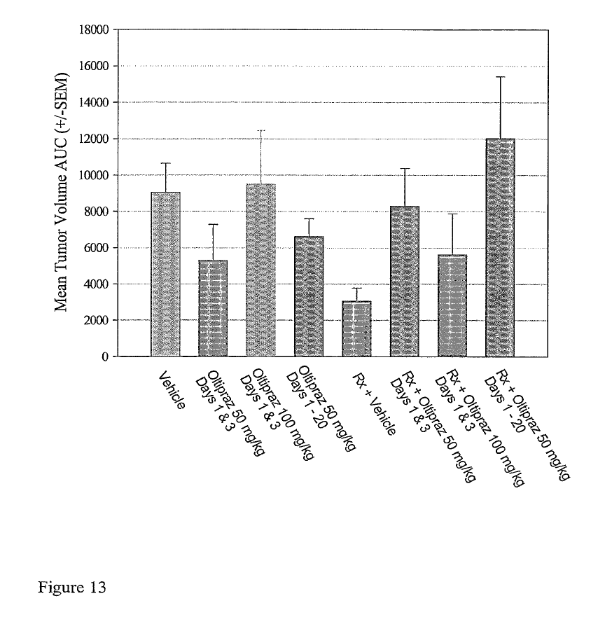

In order to evaluate the impact of 5-[2-pyrazinyl]-4-methyl-1,2-3-thione on radiation therapy, the groups receiving radiation were compared using an ANOVA on ranks analysis. No significant differences were noted between the radiation only group and the groups receiving radiation plus 5-[2-pyrazinyl]-4-methyl-1,2-3-thione (P=0.177). Individual comparisons between the radiation therapy only group and the groups receiving 5-[2-pyrazinyl]-4-methyl-1,2-3-thione plus radiation therapy using the Mann-Whitney rank sum test showed that there were no statistically significant differences between the groups (P=0.112 for the group treated with 5-[2-pyrazinyl]-4-methyl-1,2-3-thione at 50 mg/kg on days 1 and 3, P=0.977 for the group treated with 5-[2-pyrazinyl]-4-methyl-1,2-3-thione at 100 mg/kg on days 1 and 3, P=0.112 for the group treated with 5-[2-pyrazinyl]-4-methyl-1,2-3-thione at 50 mg/kg on days 1-20). The tumour volume AUC data is shown in FIG. 13.

### Discussion

In this study, the efficacy of 5-[2-pyrazinyl]-4-methyl-1,2-3-thione in inhibiting tumour growth and reducing weight loss during radiotherapy was tested using the NCI-H146 small cell lung cancer model in mice. Tumour bearing mice were treated with vehicle (0.5% CMC in water), radiation only (2 fractions of 2 Gy on days 2 and 4), 5-[2-pyrazinyl]-4-methyl-1,2-3-thione as a single agent at 50 mg/kg or 100 mg/kg on days 1 and 3 or days 1-20 or a combination of radiation plus 5-[2-pyrazinyl]-4-methyl-1,2-3-thione and radiation. 5-[2-pyrazinyl]-4-methyl-1,2-3-thione showed no evidence of toxicity in this study based on observations of survival and weight change. 5-[2-pyrazinyl]-4-methyl-1,2-3-thione as a single agent was effective in reducing tumour growth, administration of 50 mg/kg once daily on days 1 and 3 resulted in a significant reduction in tumour volume relative to vehicle controls by day 21 (P=0.030). As expected, radiation alone was effective in reducing the growth of H146 tumours (P=0.004). There was no statistically significant difference between the group receiving radiation therapy alone and the groups receiving radiation therapy plus 5-[2-pyrazinyl]-4-methyl-1,2-3-thione (P=0.177).

All documents referred to in this specification are herein incorporated by reference. Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention. Reference to any prior art in this specification is not, and should not be taken as, an acknowledgment or any form of suggestion that this prior art forms part of the common general knowledge in any country.

1-101. (canceled)

102. A pharmaceutical composition comprising 5-[2-pyrazinyl]-4-methyl-1,2-3-thione (oltipraz) or an analogue, derivative, metabolite, prodrug, solvate or pharmaceutically acceptable salt and a pharmaceutical carrier, which promotes association of the 5-[2-pyrazinyl]-4-methyl-1,2-3-thione (oltipraz) or an analogue, derivative, metabolite, prodrug, solvate or pharmaceutically acceptable salt with the outer wall of a subject's digestive tract.

103. A pharmaceutical composition of claim 102, wherein the metabolite is pyrrolopyrazine derivative metabolite 3.

104. A pharmaceutical composition of claim 102, wherein the analogue is anethole trithione ((5-(p-methoxyphenyl)-3H-1,2-dithiole-3-thio).

105. A pharmaceutical composition of claim 102, which is formulated with carboxymethyl cellulose.

106. A pharmaceutical composition of claim 102, which is formulated with cysteine or an analogue, derivative, salt or solvate thereof.

107. A pharmaceutical composition of claim 102, which is formulated with a chemotherapeutic agent.

108. A pharmaceutical composition of claim 102, which is formulated with a sulphur-containing amino acid.

109. A method for preventing or treating mucositis in a subject comprising administering to the subject a therapeutically effective amount of a composition of claim 102.

110. A method of claim 109, wherein the subject is a cancer patient.

111. A method of claim 109, wherein the cancer patient is undergoing chemotherapy, radiation treatment or a combination thereof.

112. A method of claim 109, wherein the mucositis is selected from the group consisting of: enteritis, oropharyngeal mucositis, stomatitis and proctitis.

113. A method for preventing or treating cachexia in a subject comprising administering to the subject a therapeutically effective amount of a composition of claim 102.

114. A method of claim 113, wherein the subject is a cancer patient.

115. A method of claim 114, wherein the cancer patient is undergoing chemotherapy or radiation treatment.

116. A method of claim 109, wherein the administering is oral administration.

117. A method of claim 109, wherein the composition is administered in the form of an oral rinse, in liquid form, in a capsule, in a tablet, in an injection or as a suppository.

118. A method of claim 113, wherein the administering is oral administration.

119. A method of claim 113, wherein the composition is administered in the form of an oral rinse, in liquid form, in a capsule, in a tablet, in an injection or as a suppository.
